SEQUENCE LISTING (23 sequences)

SEQ ID NO: 1 - DNA, 17850 bp, SARS-CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggc

SEQ ID NO: 2 - DNA, 20 bp, Artificial Sequence
  Synthetic Polynucleotide
tggatgatcggtggagtgta

SEQ ID NO: 3 - DNA, 20 bp, Artificial Sequence
  Synthetic Polynucleotide
cacacacgaggacaaacacc

SEQ ID NO: 4 - DNA, 20 bp, Artificial Sequence
  Synthetic Polynucleotide
aaggcacaccataaggctga

SEQ ID NO: 5 - DNA, 20 bp, Artificial Sequence
  Synthetic Polynucleotide
tcgtagagcttgccatcgtt

SEQ ID NO: 6 - DNA, 20 bp, Artificial Sequence
  Synthetic Polynucleotide
tcgaaatgaaagttccagca

SEQ ID NO: 7 - DNA, 21 bp, Artificial Sequence
  Synthetic Polynucleotide
tgtgtgagggacactccttac

SEQ ID NO: 8 - DNA, 20 bp, Artificial Sequence
  Synthetic Polynucleotide
cccaccctcctcaatgacac

SEQ ID NO: 9 - DNA, 20 bp, Artificial Sequence
  Synthetic Polynucleotide
gcactgggaatctcatggca

SEQ ID NO: 10 - DNA, 23 bp, Artificial Sequence
  Synthetic Polynucleotide
agcacagtggtgaaagagacctt

SEQ ID NO: 11 - DNA, 22 bp, Artificial Sequence
  Synthetic Polynucleotide
tccaatgcatagctggtgattt

SEQ ID NO: 12 - DNA, 19 bp, Artificial Sequence
  Synthetic Polynucleotide
cctggctcttgcttgcctt

SEQ ID NO: 13 - DNA, 21 bp, Artificial Sequence
  Synthetic Polynucleotide
ggtcttgtgtgatgttgctca

SEQ ID NO: 14 - DNA, 20 bp, Artificial Sequence
  Synthetic Polynucleotide
cagtacagccccaaaatggt

SEQ ID NO: 15 - DNA, 20 bp, Artificial Sequence
  Synthetic Polynucleotide
caagggcatatccaacaaca

SEQ ID NO: 16 - DNA, 20 bp, Artificial Sequence
  Synthetic Polynucleotide
tgttcctacccccaatgtgt

SEQ ID NO: 17 - DNA, 20 bp, Artificial Sequence
  Synthetic Polynucleotide
ggtcctcagtgtagcccaag

SEQ ID NO: 18 - protein, 742 aa, Homo sapiens
MEEGFRDRAAFIRGAKDIAKEVKKHAAKKVVKGLDRVQDEYSRRSYSRFEEEDDDDDFPAPSDGYYRGEGTQDEEEGGASSDATEGHDEDDEIYEGEYQGIPRAESGGKGERMADGAPLAGVRGGLSDGEGPPGGRGEAQRRKEREELAQQYEAILRECGHGRFQWTLYFVLGLALMADGVEVFVVGFVLPSAEKDMCLSDSNKGMLGLIVYLGMMVGAFLWGGLADRLGRRQCLLISLSVNSVFAFFSSFVQGYGTFLFCRLLSGVGIGGSIPIVFSYFSEFLAQEKRGEHLSWLCMFWMIGGVYAAAMAWAIIPHYGWSFQMGSAYQFHSWRVFVLVCAFPSVFAIGALTTQPESPRFFLENGKHDEAWMVLKQVHDTNMRAKGHPERVFSVTHIKTIHQEDELIEIQSDTGTWYQRWGVRALSLGGQVWGNFLSCFGPEYRRITLMMMGVWFTMSFSYYGLTVWFPDMIRHLQAVDYASRTKVFPGERVEHVTFNFTLENQIHRGGQYFNDKFIGLRLKSVSFEDSLFEECYFEDVTSSNTFFRNCTFINTVFYNTDLFEYKFVNSRLINSTFLHNKEGCPLDVTGTGEGAYMVYFVSFLGTLAVLPGNIVSALLMDKIGRLRMLAGSSVMSCVSCFFLSFGNSESAMIALLCLFGGVSIASWNALDVLTVELYPSDKRTTAFGFLNALCKLAAVLGISIFTSFVGITKAAPILFASAALALGSSLALKLPETRGQVLQ

SEQ ID NO: 19 - DNA, 4378 bp, Homo sapiens
gcattgccgcttccctggtgccgggagcagtcgccgctgccgcctccgcccgcggccgggacccccgtcctcgcccgggactccttacccggggaacctagaccaggtctccagaggcttgtggaagagaagcaggcgacccttcctgagttatcctggcttagcctcccaatctggctccccttccccttcccattcccctgctccccctgtcccttccccatccacccaactgaactgggtataggtcaaagctcctctccttccttttccttcctaggcactcattggctaggacctgtttgctcttttttttgtgcccagagatactggaacacgcttcatctaagtaactgtggggaggggtctttttgactctacaagtccttgagcaaaaagctgaaaaagaagcaggaggtggagaagacccagtgaagtgccccaagccccatcatggaagagggcttccgagaccgggcagctttcatccgtggggccaaagacattgctaaggaagtcaaaaagcatgcggccaagaaggtggtgaagggcctggacagagtccaggacgaatattcccgaagatcgtactcccgctttgaggaggaggatgatgatgatgacttccctgctcccagtgatggttattaccgaggagaagggacccaggatgaggaggaaggtggtgcatccagtgatgctactgagggccatgacgaggatgatgagatctatgaaggggaatatcagggcattccccgggcagagtctgggggcaaaggcgagcggatggcagatggggcgcccctggctggagtaagggggggcttgagtgatggggagggtccccctgggggccggggggaggcacaacgacggaaagaacgagaagaactggcccaacagtatgaagccatcctacgggagtgtggccacggccgcttccagtggacactgtattttgtgcttggtctggcgctgatggctgacggtgtggaggtctttgtggtgggcttcgtgctgcccagcgctgagaaagacatgtgcctgtccgactccaacaaaggcatgctaggcctcatcgtctacctgggcatgatggtgggagccttcctctggggaggtctggctgaccggctgggtcggaggcagtgtctgctcatctcgctctcagtcaacagcgtcttcgccttcttctcatcttttgtccagggttacggcactttcctcttctgccgcctactttctggggttgggattggagggtccatccccattgtcttctcctatttctccgagtttctggcccaggagaaacgaggggagcatttgagctggctctgcatgttttggatgattggtggcgtgtacgcagctgctatggcctgggccatcatcccccactatgggtggagttttcagatgggttctgcctaccagttccacagctggagggtcttcgtcctcgtctgcgcctttccttctgtgtttgccattggggctctgaccacgcagcctgagagcccccgtttcttcctagagaatggaaagcatgatgaggcctggatggtgctgaagcaggtccatgataccaacatgcgagccaaaggacatcctgagcgagtgttctcagtaacccacattaagacgattcatcaggaggatgaattgattgagatccagtcggacacagggacctggtaccagcgctggggggtccgggccttgagcctaggggggcaggtttgggggaattttctctcctgttttggtcccgaatatcggcgcatcactctgatgatgatgggtgtgtggttcaccatgtcattcagctactatggcctgaccgtctggtttcctgacatgatccgccatctccaggcagtggactacgcatcccgcaccaaagtgttccccggggagcgcgtagagcatgtaacttttaacttcacgttggagaatcagatccaccgaggcgggcagtacttcaatgacaagttcattgggctgcggctcaagtcagtgtcctttgaggattccctgtttgaagagtgttattttgaggatgtcacatccagcaacacgtttttccgcaactgcacattcatcaacactgtgttctataacactgacctgttcgagtacaagtttgtgaacagccgtctgataaacagtacattcctgcacaacaaggagggctgcccgctagacgtgacagggacgggcgaaggtgcctacatggtatactttgtgagcttcctggggacactggcagtgcttcctgggaatatcgtgtctgccctgctcatggacaagatcggcaggctcagaatgcttgctggctccagcgtgatgtcctgtgtctcctgcttcttcctgtcttttgggaacagtgagtcggccatgatcgctctgctctgcctttttggcggggtcagcattgcatcctggaatgcgctggacgtgttgactgttgaactctacccctcagacaagaggaccacagcttttggcttcctgaatgccctgtgtaagctggcagctgtgctggggatcagcatcttcacatccttcgtgggaatcaccaaggctgcacccatcctctttgcctcagctgcccttgcccttggcagctctctggccctgaagctgcctgagacccgggggcaggtgctgcagtgaaggggtctctagggctttgggattggcaggcacactgtgagaccaacaactccttccttcccctccctgccctgccatcctgacctccagagccctcactccccactccccgtgtttggtgtcttagctgtgtgtgcgtgtgcgtgtgcatgtgtgtaaaccccgtgggcagggactacagggaaggctccttcatcccagttttgagatgaagctgtactccccatttcccactgcccttgactttgcacaagagaaggctgagccccatccttctccccctgttagagaggggcccttgcttccctgttccaggggttccagaataggcttcctgccttccccatcattccctctgcctaggccctggtgaaaccacaggtatgcaattatgctaggggctggggctctggtgtagaccatggaccaaaagaacttcttagagtctgaagagtgggcctcgggtgccctctcacatctcctgttggatgctgggggagaagcaataaacctcagccctctggcctccactttcctctcaatttgggctgcaaatatgaagcctgaattttatgaaattagctttctgattcttatttattaatagattaagttctgaggcagctccgcaggactgtgtgtgaatgtgtatgtatacttacatatgtgtgtgcatgtgccatggggcggggggtatcactatactgtcctcaaatataagccaagggtaatttcagcggatgcacacacaaccctgcctcccacagttcctcccctaatctggtttctgtgttgagcctgggatggaggagccctaggccagcctgggataagagtcccacagtctagggagatctgagggcatccgacaaggcccatctccttccctcctcaagaagcagaggcctcctctggagtgagaggctccacccactacagcacaggcgggaatagcacagctgccctcccatgctccctacctgtcccctcacagggaggggagcaggggagggaaagaaaccaggcatctggtcaaaccagcagatcaaaaagcacaaagagctggggcagaggcaggaagcaggggccctcctggcagctcctctgagtggggagaggttgggcagtgagtgagggacccctaatgcagggactagaagcctcagtttccccattttacccttccacacaatagcctctgtaggttaggctgccccatcccaccctactctgtgtggctgctttctttggtgccctcccctcaccccactgtagctgtgacgtgttgtagtttttagatgtttgtaaaatgtttaaaaaaatgttaaaaggaaaaaagtgaaaataacaaaaaagaaaatcaaaattcaccttcgtcatgctgcgtccagtgccccaaccctgtggtcactctccccattttgtaacactgtaccaggtggtgactgtttaactctttggtgtctgtgctcaaaagactgccttctccagtgcccagtgtatgagtgtgtgccctgtgcccttgtccctcactccccacatgctggacgtagccctcttcctcgcacccctgggagggacccatccatctcccttgctctcctggggaaccctaaacccaactctgttgatgtgaaaaatgcagtgaaaaatattgacgaaaaataaaacggaaacaaatcctcaaaatacaaaa

SEQ ID NO: 20 - protein, 683 aa, Homo sapiens
MDDYKYQDNYGGYAPSDGYYRGNESNPEEDAQSDVTEGHDEEDEIYEGEYQGIPHPDDVKAKQAKMAPSRMDSLRGQTDLMAERLEDEEQLAHQYETIMDECGHGRFQWILFFVLGLALMADGVEVFVVSFALPSAEKDMCLSSSKKGMLGMIVYLGMMAGAFILGGLADKLGRKRVLSMSLAVNASFASLSSFVQGYGAFLFCRLISGIGIGGALPIVFAYFSEFLSREKRGEHLSWLGIFWMTGGLYASAMAWSIIPHYGWGFSMGTNYHFHSWRVFVIVCALPCTVSMVALKFMPESPRFLLEMGKHDEAWMILKQVHDTNMRAKGTPEKVFTVSNIKTPKQMDEFIEIQSSTGTWYQRWLVRFKTIFKQVWDNALYCVMGPYRMNTLILAVVWFAMAFSYYGLTVWFPDMIRYFQDEEYKSKMKVFFGEHVYGATINFTMENQIHQHGKLVNDKFTRMYFKHVLFEDTFFDECYFEDVTSTDTYFKNCTIESTIFYNTDLYEHKFINCRFINSTFLEQKEGCHMDLEQDNDFLIYLVSFLGSLSVLPGNIISALLMDRIGRLKMIGGSMLISAVCCFFLFFGNSESAMIGWQCLFCGTSIAAWNALDVITVELYPTNQRATAFGILNGLCKFGAILGNTIFASFVGITKVVPILLAAASLVGGGLIALRLPETREQVLM

SEQ ID NO: 21 - DNA, 12651 bp, Homo sapiens
gcccggatcgcccagctccgcgttagccggagctgcacgcggggctgccggggcggacgctgctctgcctgcatccggaggcggccgccagcggatttgccacccaggtcctaagactctcagtccagagagggccctgccccacacccagaaggaaggaatgctgcccagagatgaccagaggaatctagacagagcataaccttcggtggcaggacaaatcaggccagcacgcagtctgccaagtcctgctcgctccctgtcaagaaaaacagctggatccatttctaatcaacacttcccaacgcaacacttctgagtctctgaaggagaccagagcttgaaactttccagacttccaacagacatcgagtgcaaaaggatatttaggttgtctttgcacaaatctggttgatttgagagataaagggggggggaaccagtgtgactttcacctaagaagtcacatgaacatatttcacatttgaactacataatgaatgatggttattgaaatagcccaaacctctaccacagagcgagggatatagctcaaggggcaaccaggcagtcgcagaaccaaggaatggatgactacaagtatcaggacaattatgggggctatgctcccagtgatggctattaccgcggcaatgagtccaacccagaagaagatgcacagagtgatgtcaccgaaggccatgatgaggaagacgagatctatgagggcgagtaccagggtatccctcacccagatgatgtcaaggccaagcaggccaagatggcgccctccagaatggacagccttcggggccagacagacctgatggctgagaggctggaagatgaggagcagttggcccaccagtacgagaccatcatggatgagtgtggccatggccgcttccagtggatcctctttttcgtcttgggtttggccctgatggccgatggggtggaagtgttcgtggtgagttttgccctgcccagtgcagagaaggacatgtgtctgtccagttccaaaaaaggaatgctagggatgatagtctacttgggaatgatggcgggcgccttcatcctgggaggcctggctgataagctgggaaggaagcgagtcctcagcatgtctctggccgtcaatgcctccttcgcctccctctcttccttcgtgcagggatatggagccttcctcttctgccgactcatctcaggcatcggtattgggggtgctctaccgattgtttttgcctatttttctgaattcttgtctcgggagaagcgaggagaacacctcagttggctgggcatcttctggatgactgggggcctgtacgcatctgccatggcctggagcatcatcccacactatggctggggcttcagcatggggaccaattaccacttccatagctggagagtgtttgtcatcgtctgtgctctgccctgcaccgtgtccatggtggccctgaagttcatgccagagagcccaaggtttctgctagagatgggcaaacatgatgaagcctggatgattctcaagcaagtccatgacaccaacatgagagctaaggggaccccagagaaagtgttcacggtttccaacatcaaaactcccaagcaaatggatgaattcattgagatccaaagttcaacaggaacctggtaccagcgctggctggtcagattcaagaccattttcaagcaggtctgggataatgccctgtactgtgtgatggggccctacagaatgaatacactgattctggccgtggtttggtttgccatggcattcagttactatggactgacagtttggtttcctgatatgatccgctattttcaagatgaagaatacaagtctaaaatgaaggtgttttttggtgagcatgtgtacggcgccacaatcaacttcacgatggaaaatcagatccaccaacatgggaaacttgtgaatgataagttcacaagaatgtactttaaacatgtactctttgaggacacattctttgacgagtgctattttgaagacgtaacatcaacagatacctacttcaaaaattgtaccattgaatcaaccatcttttacaacacagacctctacgagcacaagttcatcaactgtcggtttatcaactccaccttcctggagcagaaggagggctgccacatggacttggagcaagataatgacttcctgatttacctcgtcagcttcctgggcagcctgtctgtcttacccgggaacatcatttctgccctgctcatggatagaattggaaggctcaagatgattggtggctccatgctaatctctgcagtctgctgcttcttcctgttttttggcaacagtgagtctgcaatgatcggctggcagtgcctgttctgtgggacaagcattgcagcctggaatgctctggatgtgatcacagtggagctgtatcccaccaaccagagagcaacagccttcggcattctcaatggattatgcaaatttggcgccatcctgggaaacaccatctttgcttcttttgttgggataaccaaagtggtccccatccttctggctgctgcttctctggttgggggtggcctgattgcccttcgactgccagagactcgagaacaggtcctgatgtgaacaacctatgggaaaaggaaaggtcgagagaatcttgtccaggacactgaaatgcatccacacttcctgcctatcacggtccggaggacaccttggatagcacgggaggagaagttgactttgtgacccctagtttaggacccacttcagctgtcaatatgtttgtaactcaggtgactgatttgggggtgccctgagccacccttagaatcacagagctgcgtgtttaacttcaagtcttcccagtccaaggcagggagaggattctccagtgagtgcacacactatgcgaggagcaagcatttctctaagtcaagtgcaaggacttaacttgcgtttgaaaaggaattagagggtcagaaacacccaggttcctccagaaagctccttggagcccaacaacttaacaaatcaacttggctggaagttagagtcattatatgaagattgggcttgaagtatatatttttgcatttaaaagtatcacctatcatattttccactcgaaaattgacatagtagcattgaggatactctgatctagaaagccaagtatttgagcaacatctatagagatctacttttctcctatgtctcctaggctttccatgataattaggtaatacatttaagaaggatatttatttctgttttgctctattcaaagaaacggaatgggatagttattctgtaaactaagtttgtatataactttatttgggtttaatttccacaactggtatctgcaaatattgccagcattttagccatattttgggagaacttggtgtttgaggtcccaggaaatgaggtctgatcaaatgaaatgcaagcacaatttcttacagccatttaactttctgttgggaggatgaattaacaaactcacattgtgcagtctgcttaatccaggcacttttctttgtgcaggtgtagtgagtagttacttctctcccttacacagatgacttgtgaaactcaagctcaccatcttcagtgctggcattttactttgccactacccaaaaacaatgtgagatgtgttcagtggcctctggtactctttgcaggcaagaatcaaacaacatggggactgagggaaggatggggaagtgtagccacagttcttccaaatgtaaatactttttgtttgttctagtggtaaaatgcaaatgcaatccatatttgttaggatggtcaggtctcatgagaaatctatgctatgtgtccagagcttttgaaacagagtccattggagtgggagttagggagtgtagtggatgccaaatatgtttttcttcagtgcttaagagaactgtttcctgaagtccagctttgaacataaacaggggtgtgggttgggggaggagcttaggacaaacctctctgatgaaggtcagcaatagactgaagtcttgactgcatggaagaggaaaaacatcagaactgtctgacaatggaggggacagtgagctacgcacaactgccagcggaggtgaacttgcacctgcccaggccggatgaacatcagcctgcaagaactagttgtttgagttgatttgcagtgctctcaatgggcaagtgccacattttccctggcagagatctccaaaaatttaaaacagaataataatggctatatcgagtgttttctcagtattggagaaatgcttaggtcctatgatagcttcgggacatctttctgtaattttcctcaattaacgggttggtaggggtaaatcttatgacacctttccaccgtcgatttgagatcagttttaatggttaaaatgtttactctccttctgtcaaccctcacctttttatttacacccctccctttttttctgtacagggagagaagacatattgactctgactggacaccctgattcctccaaatatatataccactgtgtattaatctttctctcagtgttttataggagtactaacatttattgctctgtcaataatgaaaggctcgatgtaatatagctgtaatttactttccatatgaatacagtggctaggttcataaaagagaattgtgtgagtctgggattaccacatctaaaacattattctttaatgggataatacaattcattgagcagctaccacttaaaaaacttgcaggacagttagagcctgcatttctagttaagatggatcttgtaaatttaaaattggattaacattggagtgctggggtggctgcaataatttgggggctaactccatttggtttccaagatctcacttctgcattatctttatggctctttaaaccagccacctagccaatcaagggcaattcccatctcatccatcactcaggtctttgtaaagggtgcagccaagctctgcagacttttgcaggattgtctagcctgagtaccgggctacttcttaaatgccgtcactcctgctgagataaatgcgtctttaaaaatagtctctgtggcaggtcactgggggacaatgtacagcattctggccatccacttctttttcacttcatgttctaccccaagagactcccgatgtcggctgtggagggttaaagggatgaggctttcctttgtttagcaaatctgttcacagttcttgatgatgtattttatgatgcccagcttggaaatagttgctttccatagtctcaactgtattgtgtcatctcctgatgctgatttttgatcttttgttttattaaaaataattagtgaaagaggtgtgcctatctgtgaagtttgtagtacatcatcctgaggtcatgtaacaagtaaaccccaacccagcgttccctcctacgttgtgttagttcattaaaactaaataataaaaataactgtaagaaaaccttaaggactggggttagagattattttcaggcactaaggtctcttctgtattccaaaattgattgaatcagctgggagaaacattgagaatcgcttccttcaacttttattttgcaacatggggaccttgagaggttaaacgatgtggcccagattacatgtaatgtgaggagtgggcgagaaggcctaaggtgatgctctcatgacagttctggggaagcctgcagtttggggaatgggacaatgtgacaaatactcgccattccctgagaccattcattgactattcccttgatggctttactataatgagaggaagcatcagtttaaacataaaaagattagtatttatccatccatttattcattcagcaaatccttattgagccctgaccatgggccagtctctctgccaagccctggggacacagtgatgaaaaaactggccacagtttctgcttcctgcctcattccacttgcagtgagcggatattaaattggccataaacaccgagtacgatgaccttacaatccgcaaactgcacctttcatgtgtaaaaggattgctactactttacttgtcagctgtggatttccaaatgtggtggctccctcttatttttttttctttgaggagtgtaccaattttttatctttataaaccaggtaagggaaatgatgcccttgcccattttctacagacctaatcgatttttacctaatcagttttacagaaagggttacatggaagaagagataggggccaggaatgcaagagggacattggtgagtggggtaagaatccccgtagccctgggaaaggtgtctccacttccacatctggcttttctagggggcatctgtgctaactgacctgggattatgttggatggcatatgactgcaaattcaaagaaaccaatttataataagtttatagtaagttaaaggtttgttttactctcatgtgaagggaatctggaggaaggaaagccagggctggagtggctgcctttgatcatgggagcacactccttctgcccgatcattgggagcacactccttctgcctgatcgttgggagcacactcctgcctgatcatgggcgcacgttccttctgcctgatcgttgggagcacactccttctgcctgatcatgggagcacactccttctgcctgatcattgggagcacgctccttctgcctgatcgttgggagcacactctttctgcctgatcgttgggagcacaccccttctgcctgatcattgggagcacactccttctgcctgatcatgggcacacgctccttctgcccgatcatgggcacacgctccttctgcccgatcgttgggagcacactccttctgcccaatcattgggctcacgctccttctgcccgatcgctgggctcacgctccttctgcccgatcttgggcgcacgctccttctgcccgatcgttgggcgcacgctccttctgcccgatcgttgggcgcatgctccttctgcccgatcgttgggagcacgctcattctgcccgattgttgggagcacgctccttctgcccgatcgttgggcgcacgctccttctgcccgatcgttgggcgcacgcttcttctgcctgatcgttgggcgcacgctccttctgcctgatcgttgggcgcacgctccttctgcctgatcgttgggagcacgctccttctgcctgattgttggaagcacgctccttctgccttcagactccttgatttttagtgcagtttccatcactgaggctgcctcatgggctgagatggctgttgaagcactgaacctcacatccacaattcaggcaggaagcagaaggaagggcaatgggcaaaggggctgtgccttgcagccgatccagcctccctttgctgcagaggaggcagggaaatgcggtatttactgggaacattgctgcccctcctccaaattgagggtcttggctcgaaggacaaaagccagaaaggataggtggattagtgcttcttaaacttttgcgtgcatcagaatcacccagaaagatgttaaaactcactcttcaaggccccatccccagagattcggattctggggatttggaatctggggtggggtctgaagaatctgcattttttaacaaactcccaggtgacatagatggcatcaattctcaagccatattttgagtagcactgacactcttccaataggtggtactttgacttgtcagggaagttctattgctctgataacttaaaaccaacccaaccagaagctcacaaaaactagcagcctgtgaaaaatgatcaccattttcctagagctccgagacaactagtggtagtgcaacagtaacccaaaaatgtcaatgaaaatatttccttccccagaaactgcttggtttcactcaggtcctgtccttatgggcctgtgtctggttgcagggcttgaagtatttcggaagctcagcagaggttgaaccctttcccctgggggttggtgatctttgttttggccatttccacctggtgaagattttcatagacaaatatgcttgtggcatcctgtaattcattgtgccttatgatggacctctgattctataagcttccccctagctagggatgcacgggggtgatcaagacatgactagatgtgaacctgacctgcagacagtccacctgaacgtgcctggaaatgtctcccgaaaaggatgaaaagcctcctcctttagacaaggaagacagagtggagcaaatttctctacatagatctcattcgaaaacaaatttacagaatgcagaatggctactgtgtgtcttgtgctatgctaggtctctctctctctaagtggactctgaccctgtccaaaacattgtgcttttgcatgtagctgtgtcgcatttaggcccagctctaagaaccgtttggaaataaatcacatagagcctttgattgtgaggcaggcttaaagtacattttgtttgatttgcagaggcatggtgaggaatttatatgcatggctgttgtggcagcagcgagatttccaaaaagatgaatgatgaaatgaaacagactgaagctatctcacaaatgttaaatggagaaataaaagttgttatagtcattgctaattacagaatgagcaagagatttatttagcaggaccatactatgccccctggaaggttcagtttatgagctggagcacaaggatttactgtcatttcacctgaaaacacacatttagctgtccatttacctttaatcctacaggcttaaaatactttaaaatttcagagaccattaatatgtattcctttagaaagcaaaggtctttcagaagctgtcctctaccaaagttaatgtggtcaccacatttgatgattttgctaggtagggaggtttatttaatcacagctactgtcactcaggaggatgagccatttaaaaatggtggccctgtcctccacttcttacctaccatatataatctcagtaattcagatttctctaaatcagcattagtgatcatttcaatagggtatgaacaaaaggcctataaaaatagctttccaattcgccaccaaaaatgcaaaaatgacaagtaagtgaggtaacgcatgagctaaatagcttgatttagtcactctaaaatgtgggtgtgtatatatacatacatatatatacacacacacattatgttatacaccataaatatatagaagttttacttgtcaattaaaactataaataaagtggctttccagaaaaattagatattggactctatcacaaaggaattgcataaaaatacttacaaacataactataagcaattgatgagcaaattacaaatgactgttttttattaaaacattcctccaaaagaacggcaaagcaaatcccatagacacccaaaatactaaaagtgacaaaacaatacttctttaaaaatgtattttatgatctatttattgatattctaatggttttaagaaggataattttccaaaacctaaatgctacacattttacagtggcaatatcaaccttatcatgagctaaagatttgcaatcagaacattggtagagtgcattgctgagttataacaagtgtctcaggacgaggatatttggtaggtatccttaaatatatagatgcttaccagtaggtttaatagaagatttctgaaaggtagcgcaagtttttatttcattatttttatttttatttttttgagacagagtctcactctgtcatctaggctggagtgcagtggtgcaatcttggcttgctgcaatctccacctcccaggttcaagcgattctcccacctcagcctcccgagtagctgggattacaggcatgcgccaccatgcctggctaatttttgtatttttagtagagacggggtttcgccatgttggccaggctcgtctcgaactcctggcctcaagtgatctgcccgcctcagcctcccaaagtgctgggattacaaatgtgagctactatgcctggtcccaacttcttaaaattaaattttggggaagttcatggagatatttttgacatgtaaaagtagaaaacaaaccatccatagtgtgtgcatatatacacacaaggtttggtttaatatacttgctgtgcactagtcagttttgattcaggttacataagggataaaatatagaaagacattttaaagtttgaaatatgtgctaagtagttagagatttggcagttttgccacatttcaaatgggccaaacattaaaataacttatccatgaatttcaggtttcaaggaaggaggaatatctgactatttttttttgtgtgtgagatttggtctcagctttttggtccatctgaatcctggaagtccccaactctaccagaccaaaagggtgtctctattttttgttgttacttctctgttcttgatgtaatgagttaagtagatgaaagagattaaggtgcagggaagggctcttaaaagtttaatgtttcagctttccatagtcattcatacttgtgaatcttcttcctcttctacttcaaagtagccaaaacttggaaagttcaaattaatctttgggctccaaagagcaattctgaaggattaacaggtttcattctctccagtgggaggtggcctagacaggtgccggttttggggagaggtgagagtacccagatgtgggtgactatttcctgttcttccctgaaagtcaggccagtggtctcgaatcattcccacagcaactgctacaaaagtgactatcttaatcctttggctagatgggatacctcttctaccttttcagaggtgagctgttgagtagtaatgtccgctacaacaccacgctcccggaatggtggttcctaagagcaaagtcagttaatacaatttctccaggtatccagctatctccaggtgtgcggtcctgtgaagcactctaagaactgtaaaaaatatttcataggtatagaaggcatttgtaatactgttggttcaaatgtgctgacatgtagccagtggaggaagatgtttctttggaagcctgccatctttctgtaacgcaccttcaacctgagataattaagagtcattacgtttcccagtcccatgtcacattgaccagtcctgtggtcctatggctaagcggaggacacggccacaagctgtgagtcatgaaaaggagggtgcaggtgataggcaaagggacccattagccagtttacaatgactggcaactgaccagatagagcttaccgtatgtccatgtagagttgctcatattagtctgtaaataattctatgcatagtgaatgcagcttctgaattattaaactgatgctataaatgtgaactgacaaaacccacttttggtctctcttcatatttttcgtggtttcatttatctctcccttcagatgtttttccatctgactgtcactggatagtggaattgctctgattaccgatgccttttttgttctttggtatattaaaatagtagtaacagttgatctggtgattggtgaatacagtttaatctttaataccttgtgatccagatatttaacaaacacacatgaaatttactgaatgacaagaactgtgataaagacttttaaagcctatcccctttaaccttcataataacctaatgagtaagtgttattttcttccctttagaagtggggaaactggaacacaggaaagtcacttgtcaaaggccaaatagctacagactgagtattccttatcccaaatgtttgggaccagaagtgttctggatttccggttttaaaagattttgggatattggcattatacctaccagctgagcatcccaagtctgcacatttgatatctaaaatactccagtgagcttttccttggagtacacgttggcgctcaaaaagttttcaatttgggagcatttcggatttttgacttttggatttggatttggaatgctcaacctgttttcagagtcagagccaagttctgaatgctcatcacctggcatatagtatggtataatcaccccaccaaacctcttccaaaagaagccagggtcatctgctgcagatgtttctgggccactaggccaatattaaccaaattattatagacaccatcttaatgtagaggatcccacaagactgtgacctaataaaaccctcattttccctatgcattgtgactaacacagtatttgatacgtagtaggtctcaatagatgatgaatagaccgatgaatgagcacatgagttgaatggtgaggagcaagtgaagtgtcatatgtaaaagagttaattctcagattgattcaaatgcggacttaacactgcagctgtaactctgggtccgggtcttggtggctcctgctttgctgagctttaaaactttagtaacctgatgatggagagatctggggggtgcagaggagggacctggttctggagtggtgatgctggttgtcagagggctgagggtggtggccttttgctaactggtatggaagcatttcggtattttaacaagtatgaaagtagctatgtgaatttgccaaatagaaatcttggaggccaggtgcagtggttcacacctgtaatcccagcattttggg

SEQ ID NO: 22 - protein, 676 aa, Homo sapiens
MEDSYKDRTSLMKGAKDIAREVKKQTVKKVNQAVDRAQDEYTQRSYSRFQDEEDDDDYYPAGETYNGEANDDEGSSEATEGHDEDDEIYEGEYQGIPSMNQAKDSIVSVGQPKGDEYKDRRELESERRADEEELAQQYELIIQECGHGRFQWALFFVLGMALMADGVEVFVVGFVLPSAETDLCIPNSGSGWLGSIVYLGMMVGAFFWGGLADKVGRKQSLLICMSVNGFFAFLSSFVQGYGFFLFCRLLSGFGIGGAIPTVFSYFAEVLAREKRGEHLSWLCMFWMIGGIYASAMAWAIIPHYGWSFSMGSAYQFHSWRVFVIVCALPCVSSVVALTFMPESPRFLLEVGKHDEAWMILKLIHDTNMRARGQPEKVFTVNKIKTPKQIDELIEIESDTGTWYRRCFVRIRTELYGIWLTFMRCFNYPVRDNTIKLTIVWFTLSFGYYGLSVWFPDVIKPLQSDEYALLTRNVERDKYANFTINFTMENQIHTGMEYDNGRFIGVKFKSVTFKDSVFKSCTFEDVTSVNTYFKNCTFIDTVFDNTDFEPYKFIDSEFKNCSFFHNKTGCQITFDDDYSAYWIYFVNFLGTLAVLPGNIVSALLMDRIGRLTMLGGSMVLSGISCFFLWFGTSESMMIGMLCLYNGLTISAWNSLDVVTVELYPTDRRRRVLPCCPG

SEQ ID NO: 23 - DNA, 3041 bp, Homo sapiens
accactccgcgcgctgcaggcgagagtggcagacggaggcagcccggggaagcgagccggagcggcgcccgcactgaggcggctgcagtgctgacaccactcagggcaagggtgtccgacggctggagcgttctgttttgaacccaaagtggatgatgctgtcagagctgaaccactgaaaggaggctgtgaaaatttcccatcttctcattggccatcagttgagataagatggaagactcttacaaggataggacttcactgatgaagggtgccaaggacattgccagagaggtgaagaaacaaacagtaaagaaggtgaatcaagctgtggaccgagcccaggatgaatacacccagaggtcctacagtcggttccaagatgaagaagatgatgatgactactacccggctggagaaacctataatggtgaggccaacgatgacgaaggctcaagtgaagccactgaggggcatgatgaagatgatgagatctatgagggggagtatcagggcatccccagtatgaaccaagcgaaggacagcatcgtgtcagtggggcagcccaagggcgatgagtacaaggaccggcgggagctggaatcagaaaggagagctgacgaggaagagttagcccagcagtatgagctgataatccaagaatgcggtcatggtcgttttcagtgggcccttttcttcgtcctgggcatggctcttatggcagacggtgtagaggtgtttgtcgttggcttcgtgttacccagtgctgagacagacctctgcatcccaaattcaggatctggatggctaggcagcatagtgtacctcgggatgatggtgggggcgttcttctggggaggactggcagacaaagtgggaaggaaacagtctcttctgatttgcatgtctgtcaacggattctttgccttcctttcttcatttgtccaaggttatggcttctttctcttctgtcgcttactttctggattcgggattggaggagccatacccactgtgttctcgtactttgctgaagtcctggcccgggaaaagcggggcgaacacttgagctggctctgcatgttctggatgatcggtggcatctacgcctctgccatggcctgggccatcatcccgcactacgggtggagcttcagcatgggatcggcctaccagtttcacagttggcgtgtgtttgtcatcgtctgtgcactcccctgtgtctcctccgtggtggccctcacattcatgcctgaaagcccacgattcttgttggaggttggaaaacatgatgaagcttggatgattctgaagttaattcatgacaccaacatgagagcccggggtcagcctgagaaggtcttcacggtaaacaaaataaaaactcctaaacaaatagatgagctgattgaaattgagagtgacacaggaacatggtataggaggtgttttgttcggatccgcaccgagctgtacggaatttggttgacttttatgagatgtttcaactacccagtcagggataatacaataaagcttacaattgtttggttcaccctgtcctttgggtactatggattatccgtttggttccctgatgtcattaaacctctgcagtccgatgaatatgcattgctaaccagaaatgtggagagagataaatatgcaaatttcactattaactttacaatggaaaatcagattcatactggaatggaatacgacaatggcagattcataggggtcaagttcaaatctgtaactttcaaagactctgtttttaagtcctgcacctttgaggatgtaacttcagtgaacacctacttcaagaactgcacatttattgacactgtttttgacaacacagattttgagccatataaattcattgacagtgaatttaaaaactgctcgttttttcacaacaagacgggatgtcagattacctttgatgatgactatagtgcctactggatttattttgtcaactttctggggacattggcagtattgccagggaacattgtgtctgctctgctgatggacagaattgggcgcttaacaatgctaggtggctctatggtgctttcggggatcagctgtttcttcctttggttcggcaccagtgaatccatgatgataggcatgctgtgtctgtacaatggattgaccatctcagcctggaactctcttgacgtggtcactgtggaactgtaccccacagaccggagacgcagagtcttgccatgttgccccggctgatcttgaactcctggcctcaagcaatcctcctgcctcagcctcccaaagttctgggattacagtctcgagccactgcacctggctttctttccatttttgaggattaaaattacttgaaattttccaaatatattatacaaggtcttgctaagaacttagtagatttctttattgttgtcttttttctctctcttttgccttgtagaaatatcccacaaagtgaagtaaccaagtcaacaagtgtgaacaattttatagctctccttaaatttactatacagctctcagaaacaacggagccagtttatagcaggagccattagtgggtgtggacctcttttctaagatctgcttctaaacttggctttatcacataaagagagaaaagcattacaaacaatttgaaggaattctttgtcttataaaaaaaattctgaaaacctccttgtattcccactaggaggtatagttaaccttgcaactccacaaacagaccactttacacacttgcccaggggcctttgtttttgcttgcccctcctctgaaatgctctctacaggtgattaagtggcttttccctttacttaggtgtctactcaggtaaaatcttaaccaagacgcctttacttaaaatgtccctctgccccaccctgaggcttttaacttgcatagccctcatcatcacctgacataatatgtacttctttgcttttatagatattatctgtgtccctccactaggatgtccatttcatgggaataaagactttgtctcttt

(19) United States
(12) Patent Application Publication
(10) Pub. No.: US 2024/0423953 A1
(43) Pub. Date: Dec. 26, 2024

(54) METHODS AND COMPOSITIONS FOR TARGETING SV2 PROTEINS FOR IMMUNE REGULATION

(71) Applicant: THE REGENTS OF THE UNIVERSITY OF CALIFORNIA, Oakland, CA (US)

(72) Inventor: Ke SHUAI, Calabasas, CA (US)

(73) Assignee: THE REGENTS OF THE UNIVERSITY OF CALIFORNIA, Oakland, CA (US)

(21) Appl. No.: 18/262,890

(22) PCT Filed: Jan. 25, 2022
(86) PCT No.: PCT/US2022/013726

§ 371 Date: Jul. 25, 2023

Related U.S. Application Data
(60) Provisional application No. 63/141,778, filed on Jan. 26, 2021.

(51) Int. Cl. A61K 31/4015; A61P 37/06; C12N 15/113

(52) U.S. Cl. CPC A61K 31/4015; A61P 37/06; C12N 15/113; C12N 2310/11; C12N 2310/14; C12N 2310/531

(57) ABSTRACT

Disclosed here are methods and compositions for targeting SV2 proteins, including SV2A, SV2B, and SV2C. Certain aspects include methods for treatment of conditions associated with group 2 innate lymphoid cells (ILC2s) including, for example, autoimmune disorders, COVID-19 pneumonia, obesity. and cancer. Also disclosed are methods for reducing expression of a cell surface receptor by reducing expression of an SV2 protein in the cell.

This application claims benefit of priority of U.S. Provisional Application No. 63/141,778, filed Jan. 26, 2021, which is hereby incorporated by reference in its entirety.

### SEQUENCE LISTING

The instant application contains a Sequence Listing which has been submitted in ASCII format and is hereby incorporated by reference in its entirety. Said ASCII copy, created on Jan. 18, 2022, is named UCLA.P0131WO_Sequence_Listing.txt and is 71,226 bytes in size.

### BACKGROUND

### I. Field of the Invention

Aspects of this invention relate to at least the fields of molecular biology, immunology, and medicine.

### II. Background

Recent studies suggest an important role of the alarmin cytokine IL-33 in the immunopathology of COVID-19, and clinical trials are currently underway to evaluate the therapeutic benefits of IL-33 signaling blockers for the treatment of COVID-19 pneumonia¹⁻⁵. IL-33 signals through its receptor ST2 to promote the differentiation of group 2 innate lymphoid cells (ILC2s), which are associated with various conditions including immune disorders and cancer⁶⁻¹⁵. No therapeutic drugs targeting ILC2s have been approved. There is a need for effective therapeutics capable of targeting ILC2s (e.g., via direct or indirect modification of IL-33 signaling) for treatment of ILC2-associated conditions.

### SUMMARY

Aspects of the present disclosure address the needs of patients for clinical treatment by providing methods and compositions capable of targeting ILC2s via inhibition of IL-33 signaling through SV2A-mediated regulation of the IL-33 receptor, ST2. Also disclosed are methods and compositions for targeting SV2 proteins, including SV2A, for regulation of cell surface proteins. Certain embodiments are directed to levetiracetam and/or brivaracetam and methods of use thereof for SV2 protein targeting and treatment of ILC2-associated conditions.

Embodiments of the disclosure include methods for treatment of a condition associated with group 2 innate lymphoid cells (ILC2s), methods for treatment of cancer, methods for treatment of COVID-19 pneumonia, methods for treatment of an autoimmune or inflammatory condition, methods for treatment of an allergic disorder, methods for reducing an amount of a cell surface receptor, methods for reducing expression of a cell surface receptor, methods for increasing an amount of a cell surface receptor, methods for increasing expression of a cell surface receptor, methods for reducing an amount of an SV2 protein, methods for reducing expression of an SV2protein, methods for increasing an amount of an SV2 protein, methods for increasing expression of an SV2 protein, methods for increasing an expression level of an SV2 mRNA, methods for decreasing an expression level of an SV2 mRNA, methods for reducing a number of ILC2s in a subject, and methods for reducing an inflammatory response in a subject. One or more of these aspects may be excluded in certain embodiments. Methods of the present disclosure can include at least 1, 2, 3, 4, or more of the following steps: administering an agent that reduces an amount of an SV2 protein, administering an agent that reduces expression of an SV2 protein, administering an SV2 protein-targeting agent, administering an SV2A-targeting agent, administering an SV2B-targeting agent, administering an SV2C-targeting agent, administering levetiracetam, administering brivaracetam, reducing an amount of a cell surface protein, reducing expression of a cell surface protein, reducing an amount of an SV2 protein, reducing expression of an SV2protein, diagnosing a subject with cancer, diagnosing a subject with an autoimmune or inflammatory disorder, diagnosing a subject with COVID-19 pneumonia, administering a cancer therapy, and administering an anti-inflammatory agent. One or more of these steps may be excluded in certain embodiments.

Disclosed herein, in some aspects, is a method for treating a subject for a condition associated with group 2 innate lymphoid cells (ILC2s), the method comprising administering to the subject an effective amount of an agent that reduces an amount of a synaptic vesicle glycoprotein 2 (SV2) protein. In some embodiments, the condition is an autoimmune condition. In some embodiments, the condition is an allergic disorder. In some embodiments, the condition is cancer. In some embodiments, the condition is pneumonia. In some embodiments, the condition is COVID-19 pneumonia. In some embodiments, administering the effective amount of the agent reduces an amount of a cell surface protein in cells of the subject. In some embodiments, the cell surface protein is a cell surface receptor. In some embodiments, the cell surface protein is ST2L. In some embodiments, the cell surface protein is ACE2. In some embodiments, the cell surface protein is PD-1. In some embodiments, administering the effective amount of the agent reduces a number of ILC2s in the subject. At least 1, 2, 3, or more agents that reduce an amount of an SV2protein may be administered to the subject. The disclosed methods may comprise reducing the amount of one or more SV2 proteins (e.g., SV2A) by at least, at most, or about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 99.1%, 99.2%, 99.3%, 99.4%, 99.5%, 99.6%, 99.7%, 99.8%, or 99.9%, or any range or value derivable therein, compared to an untreated subject.

Also disclosed, in some embodiments, is a method for reducing an amount of a cell surface receptor in a cell comprising administering to the cell an agent that reduces an amount of an SV2 protein in the cell. In some embodiments, the cell surface receptor is ST2L. In some embodiments, the cell surface receptor is not ST2L. In some embodiments, the cell surface protein is ACE2. In some embodiments, the cell surface protein is PD-1. Methods of the disclosure may comprise reducing an amount of at least 1, 2, 3, 4, or more cell surface receptors in a cell. The disclosed methods may comprise reducing the amount of one or more cell surface proteins by at least, at most, or about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 99.1%, 99.2%, 99.3%, 99.4%, 99.5%, 99.6%, 99.7%, 99.8%, or 99.9%, or any range or value derivable therein, compared to an untreated cell.

Also disclosed, in some embodiments, is a method for increasing an amount of a cell surface receptor in a cell comprising administering to the cell an agent that increasing an amount of an SV2 protein in the cell. In some embodiments, the cell surface receptor is ST2L. In some embodiments, the cell surface receptor is not ST2L. In some embodiments, the cell surface protein is ACE2. In some embodiments, the cell surface protein is PD-1. Methods of the disclosure may comprise increasing an amount of at least 1, 2, 3, 4, or more cell surface receptors in a cell. The disclosed methods may comprise increasing the amount of one or more cell surface proteins by at least, at most, or about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 100%, 110%, 120%, 130%, 140%, 150%, 160%, 170%, 180%, 190%, 200%, 300%, 400%, or 500%, or any range or value derivable therein, compared to an untreated cell.

In some embodiments, the SV2 protein is SV2A. In some embodiments, the SV2 protein is SV2B. In some embodiments, the SV2 protein is SV2C. In some embodiments, the agent increases a rate of degradation of the SV2 protein. In some embodiments, the agent decreases an expression level of an SV2 mRNA encoding the SV2 protein. In some embodiments, the agent is a nucleic acid targeting agent. In some embodiments, the agent is an siRNA, an shRNA, or an antisense oligonucleotide. In some embodiments, the agent is an SV2A-binding agent. In some embodiments, the agent is levetiracetam or a derivative thereof. In some embodiments, the agent is levetiracetam. In some embodiments, the agent is brivaracetam or a derivative thereof. In some embodiments, the agent is brivaracetam. In some embodiments, the agent is padsevonil. It is specifically contemplated that any one or more of the disclosed agents may be excluded from certain embodiments.

Disclosed herein, in certain aspects, is a method for treating a subject for COVID-19 pneumonia, the method comprising administering to the subject an effective amount of levetiracetam or brivaracetam. In some embodiments, the subject has one or more symptoms of pneumonia. In some aspects, disclosed is a method for treating a subject for cancer, the method comprising administering to the subject an effective amount of levetiracetam or brivaracetam. Also disclosed is a method for treating a subject for an autoimmune disorder, the method comprising administering to the subject an effective amount of levetiracetam or brivaracetam. In some embodiments, administering to the subject the effective amount of levetiracetam or brivaracetam reduces an amount of an SV2 protein in cells of the subject. In some embodiments, administering the effective amount of levetiracetam or brivaracetam reduces the amount of the SV2 protein in the subject by at least, at most, or about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 99.1%, 99.2%, 99.3%, 99.4%, 99.5%, 99.6%, 99.7%, 99.8%, or 99.9%, or any range or value derivable therein, compared to an untreated subject. In some embodiments, the SV2 protein is SV2A. In some embodiments, the SV2 protein is SV2B. In some embodiments, the SV2 protein is SV2C. In some embodiments, administering to the subject the effective amount of levetiracetam or brivaracetam reduces a number of ILC2s in the subject. In some embodiments, the method comprises administering to the subject an effective amount of levetiracetam or a derivative thereof. In some embodiments, the method comprises administering to the subject an effective amount of levetiracetam. In some embodiments, the method comprises administering to the subject an effective amount of brivaracetam or a derivative thereof. In some embodiments, the method comprises administering to the subject an effective amount of brivaracetam.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

“Individual, “subject.” and “patient” are used interchangeably and can refer to a human or non-human.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

A variety of embodiments are discussed throughout this application. Any embodiment discussed with respect to one aspect applies to other aspects as well and vice versa. Each embodiment described herein is understood to be embodiments that are applicable to all aspects. It is contemplated that any embodiment discussed herein can be implemented with respect to any method or composition, and vice versa. Furthermore, compositions and kits can be used to achieve methods disclosed herein.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C show analysis of Sv2a expression in the lungs and brain. FIG. 1A shows Q-PCR analysis of Sv2a in the lungs and brain from C57/BL6 mice. FIG. 1B shows immunoblot analysis of brain protein samples from WT and Sv2a-pups at the age of 12 days using a monoclonal anti-SV2A antibody. FIG. 1C shows immunoblot analysis of SV2A amount in the lungs and brain of C57/BL6 mice at various concentrations as indicated.

FIGS. 2A-2G show data demonstrating the regulation of IL-33 signaling by SV2A. FIG. 2A shows immunoblot analysis of SV2A protein in lung or brain protein extracts prepared from mice intranasally injected with PBS or IL-33 (100 ng) for 4 hr, demonstrating that IL-33 treatment suppressed SV2A protein in the lungs, but not in the brain. Shown are representative results of at least three independent experiments. FIG. 2B shows immunoblot analysis of SV2A protein expression in the lungs of Sv2a^{+/+}, Sv2a^{+/-}, and Sv2a^{-/-} pups of day 12 age. FIG. 2C shows the expression of ST2L and sST2 in the same lung samples as in FIG. 2B. FIG. 2D shows immunoblot analysis of ST2L in WT and Sv2a KO primary MEFs treated with CHX for the indicated times. FIG. 2E shows flow cytometry analysis of the cell surface expression of ST2L in WT and Sv2a KO primary MEFs treated with CHX. FIG. 2F shows immunoblot analysis of ST2L expression in WT and Sv2a KO primary MEFs treated with IL-33 for the indicated times. FIG. 2G shows Q-PCR analysis of Il13 and Il5 gene induction in WT and Sv2a KO primary MEFs treated with IL-33 for the indicated times.

FIG. 3 shows Q-PCR analysis of lung Sv2a gene expression in response to IL-33 stimulation. Sex-and age matched C57/BL mice were intranasally injected with either PBS or IL-33 (100 ng) for 4 hrs, lung tissues were harvested for Q-PCR analysis for Sv2a gene. Shown are representative results from two pairs of mice. Error bars represent SD.

FIGS. 4A-4D show Q-PCR analysis of Sv2a (FIG. 4A), St21 (FIG. 4B), sSt2 (FIG. 4C), and Il1rap (FIG. 4D) in the lungs of Sv2a^{+/+}, Sv2a^{-/-}, Sv2a^{-/-} pups. Lung RNA samples were prepared from Sv2a^{+/+}, Sv2a^{+/-}, Sv2a^{-/-} pups at the age of 12 days. Shown is the representative result from at least 3 independent experiments. Error bars represent SD.

FIGS. 5A-5E show data demonstrating regulation of ILC2 responses by SV2A. FIG. 5A shows immunoblot analysis of SV2A expression in the lungs and brain of Sv2a^{+/+} and Sv2a^{+/-} young adult mice. FIG. 5B shows immunoblot analysis of ST2L expression in the lungs of Sv2a^{+/+} and Sv2a^{+/-} young adult mice intranasally injected with PBS or IL-33 for the indicated times. FIGS. 5C-5E show analysis of Sv2a heterozygosity on ILC2 responses. Sex-matched Sv2a^{+/+} and Sv2a^{+/-} littermates were intranasally injected with IL-33 (100 ng) daily on day 1, 2, and 3, and lung tissues (FIGS. 5C and 5D) or BM samples (FIG. 5E) were harvested on day 4. The frequency and numbers of lung ILC2s (CD45⁺Lin−KLRG1⁺CD90⁺), and the geometric mean fluorescence intensity (GMFI) of ST2 in lung ILC2s were analyzed by flow cytometry (FIG. 5C). FIG. 5D; same as in FIG. 5C except lung IL13⁺ILC2s or IL5⁺ILC2s were analyzed. FIG. 5E; same as in FIG. 5C except the frequency and numbers of BM ILC2P (CD45⁺Lin⁻CD127⁺CD25⁺) were analyzed by flow cytometry. n=6 mice/group. Error bars represent SD. P values were determined by non-paired t-test. *:≤0.05; **:≤0.01.

FIGS. 6A-6C show gating strategies for lung ILC2s and BM ILC2P. Lung ILC2s were gated as CD45⁺Lin⁻KLRG1⁺CD90⁺ (FIG. 6A) or CD45⁺Lin⁻GATA3⁺CD90⁺CD25⁺ (FIG. 6B), while BM ILC2P was gated as CD45⁺Lin⁻CD127⁺CD25⁺ (FIG. 6C). Pregate: singlet, live, CD45⁺ cells.

FIGS. 7A and 7B show analysis of Sv2a heterozygosity on ILC2 responses. Sex-matched Sv2a^{+/+} and Sv2a^{+/-} littermates were intranasally injected with IL-33 (100 ng) daily on day 1, 2, and 3, and lung tissues were harvested at day 4. The frequency (FIG. 7A) and numbers (FIG. 7B) of lung ILC2s (CD45⁺Lin⁻GATA3⁺CD90⁺CD25⁺) were analyzed by flow cytometry (n=6 mice/group). Error bars represent SD. P values were determined by non-paired t-test. *:≤0.05; **:≤0.01.

FIGS. 8A-8E show data demonstrating that LEV destabilize SV2A protein in the lungs, but not in the brain. FIGS. 8A and 8B show immunoblot analysis of SV2A expression in the lungs (FIG. 8A) and brain (FIG. 8B) of LEV-treated mice. FIG. 8C shows immunoblot analysis of SV2A protein expression in lung protein extracts in the absence or presence of various concentrations of LEV incubated at 37° C. as indicated. FIG. 8D; same as in FIG. 8C except brain protein extracts were used. FIG. 8E shows immunoblot analysis from brain extracts from WT mice mixed with either lung or brain protein extracts from Sv2a^{-/-} pups of day 12 age, with the reaction mixtures containing various concentrations of LEV as indicated. Shown is a representative result from at least 3 independent experiments.

FIGS. 9A-9C show analysis of Sv2a heterozygosity on ILC2 responses in untreated mice. Lung tissues from sex-matched Sv2a^{+/+} and Sv2a^{+/-} littermates were harvested and the frequency (FIG. 9A) and numbers (FIG. 9B) of lung ILC2s (CD45⁺Lin⁻KLRG1⁺CD90⁺) were analyzed by flow cytometry (n=4 mice/group). FIG. 9C; same as in FIG. 9A except the frequency of BM ILC2P (CD45⁺Lin⁻CD127⁺CD25⁺) were analyzed by flow cytometry (n=4 mice/group). Error bars represent SD. P values were determined by non-paired t-test. *:<0.05; **:≤0.01.

FIGS. 10A-10E show data demonstrating that LEV treatment inhibits ILC2 responses. FIG. 10A shows immunoblots were analyzed using anti-STL antibody as indicated, demonstrating that intranasal injection of LEV for 4 hr or daily for three consecutive days (3D) reduced ST2L expression in the lungs of sex- and age-matched C57/BL6 naïve mice. FIGS. 10B-10D show analysis of LEV treatment on ILC2 responses. Sex- and age-matched C57/BL6 young adult mice were intranasally injected with LEV (17 mg/kg) daily on day 1, 2, and 3, and lung tissues (FIG. 10B and 10C) or BM samples (FIG. 10D) were harvested on day 4. The frequency and numbers of lung ILC2s and the geometric mean fluorescence intensity (GMFI) of ST2 in lung ILC2s were analyzed by flow cytometry (n=5 mice/group). (FIG. 10B). FIG. 10C; same as in FIG. 10B except lung IL13⁺ILC2s or IL5⁺ILC2s or IL5⁺IL13⁺ILC2s were analyzed (n=5 mice/group). FIG. 10D; same as in FIG. 10B except the frequency of BM ILC2Ps were analyzed by flow cytometry (n=6 mice/group). FIG. 10E shows analysis of LEV treatment on ILC2 responses in a papain-induced lung inflammation model. Sex- and age-matched C57/BL6 naive mice were intranasally injected with papain alone (PN) or together with LEV (PN+LEV) daily on day 1, 2, and 3, and lung tissues samples were harvested on day 4. PN: 50 μg; LEV: 17 mg/kg. The frequency of lung ILC2s, IL13⁺ILC2, IL5⁺ILC2, and IL5⁺IL13⁺ILC2s were analyzed by flow cytometry (n=9 mice/group). Error bars represent SD. P values were determined by non-paired t-test. *:≤0.05; **:≤0.01. ****:≤0.0001.

FIG. 11 shows Q-PCR analysis of lung Sv2a gene expression in response to LEV stimulation. Sex-and age matched C57/BL mice were intranasally injected with either PBS or LEV (17 mg/kg of body weight) for 4 hrs, lung tissues were harvested for Q-PCR analysis for Sv2a gene. Shown is a representative result from at least 3 independent experiments. Error bars represent SD.

FIGS. 12A-12D show results from analysis of Sv2b^{+/-} mice following IL-33 treatment, as described in Example 2. FIG. 12A shows frequency of lung ILC2s in Sv2b^{+/+} (+/+) and Sv2b^{+/-} (+/−) mice. FIG. 12B shows number of lung ILC2s in Sv2b^{+/+} (+/+) and Sv2b^{+/-} (+/−) mice. FIG. 12C shows ST2 expression, measured by flow cytometry, in lung ILC2s from Sv2b^{+/+} (+/+) and Sv2b^{+/-} (+/−) mice. FIG. 12D shows frequency of IL5⁺IL13⁺ILC2s in Sv2b^{+/+} (+/+) and Sv2b^{+/-} (+/−) mice.

### DETAILED DESCRIPTION OF THE INVENTION

The synaptic vesicle glycoprotein 2A (SV2A) plays a key role in epilepsy pathophysiology. Levetiracetam (LEV) is a FDA-approved anti-seizure drug that targets SV2A. The molecular basis of SV2A function and the mechanistic effect of LEV binding are unclear. The present disclosure is based at least in part on the discovery that SV2A regulates IL-33 signaling and ILC2 responses by modulating ST2. LEV administration was discovered to unexpectedly trigger the degradation of lung SV2A protein and suppress ILC2 responses. Aspects of the present disclosure identify SV2A as a novel druggable target of IL-33 signaling and ILC2s and indicate that LEV and related compounds (e.g., brivaracetam, padsevonil) may be used for the regulation of ST2 expression and for the treatment of IL-33/ILC2-associated diseases including allergic disorders, pneumonia (e.g., COVID-19 pneunomia), obesity, and cancer.

### I. SV2 Protein Targeting

Disclosed herein, in some embodiments, are methods and compositions for targeting one or more SV2 proteins. Certain aspects are directed to SV2 protein-targeting agents. As used herein, an “SV2 protein-targeting agent” describes an agent (pharmaceutical agent, biological agent, etc.) capable of modifying the amount (also the “protein expression”) of an SV2 protein in a cell. In some embodiments, the SV2 protein-targeting agent modifies the amount of the SV2protein by modifying expression of SV2 mRNA. In some embodiments, the SV2 protein-targeting agent modifies the amount of the SV2 protein without any modification of SV2 mRNA expression, such as by increasing or decreasing a rate of degradation of an SV2 protein.

In some embodiments, an SV2 protein-targeting agent of the disclosure is an agent that increases the amount of an SV2 protein in a cell. In some embodiments, an SV2 protein-targeting agent of the disclosure increases the amount of an SV2 protein by decreasing a rate of degradation of the SV2 protein (i.e., by stabilizing the protein). An SV2-protein targeting agent of the disclosure may increase the amount of an SV2 protein in a cell or subject by at least, at most, or about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 100%, 110%, 120%, 130%, 140%, 150%, 160%, 170%, 180%, 190%, 200%, 300%, 400%, or 500%, or any range or value derivable therein, compared to an untreated cell or subject.

In some embodiments, an SV2 protein-targeting agent of the disclosure is an agent that reduces the amount of an SV2 protein in a cell. In some embodiments, an SV2 protein-targeting agent of the disclosure reduces the amount of an SV2 protein by increasing a rate of degradation of the SV2 protein. An SV2-protein targeting agent of the disclosure may reduce the amount of an SV2 protein in a cell or subject by at least, at most, or about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 99.1%, 99.2%, 99.3%, 99.4%, 99.5%, 99.6%. 99.7%, 99.8%, or 99.9%, or any range or value derivable therein, compared to an untreated cell or subject.

An SV2 protein-targeting agent of the disclosure may be an SV2A-targeting agent (i.e., an agent that modifies the amount of SV2A). An SV2 protein-targeting agent of the disclosure may be an SV2B-targeting agent (i.e., an agent that modifies the amount of SV2B). An SV2 protein-targeting agent of the disclosure may be an SV2C-targeting agent (i.e., an agent that modifies the amount of SV2C).

In some embodiments, an SV2 protein-targeting agent is a nucleic acid targeting agent. A “nucleic acid targeting agent” describes a nucleic acid configured to modify the expression of an SV2 mRNA. A nucleic acid targeting agent of the disclosure may be, for example, a short hairpin RNA (shRNA), short interfering RNA (siRNA), or antisense oligonucleotide configured to bind to SV2A mRNA, SV2B mRNA, and/or SV2C mRNA, thereby modifying expression of SV2A, SV2B, and/or SV2C. In some embodiments, a nucleic acid targeting agent is an oligonucleotide (e.g., siRNA, shRNA, antisense oligonucleotide, etc.) having a length of at least or at most 10, 11. 12. 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides, or any range derivable therein. In some embodiments, a nucleic acid targeting agent is an oligonucleotide having a length of 19, 20, 21, 22, 23, 24, or 25nucleotides, or any range derivable therein. In some embodiments, the nucleic acid targeting agent is an shRNA molecule. In some embodiments, the nucleic acid targeting agent is an siRNA molecule. In some embodiments, the nucleic acid targeting agent is an antisense oligonucleotide molecule. A nucleic acid targeting agent of the disclosure may be administered to a cell or subject as a “naked” nucleic acid molecule, or via a delivery system or composition such as, for example, nanoparticles, liposomes, exosomes, viral vectors, or other suitable system for delivery of a nucleic acid molecule to a cell or subject. Methods and systems for delivery of nucleic acid targeting agents include those described in, for example, Dammes N. Peer D. Trends Pharmacol Sci. 2020 October;41 (10): 755-775, incorporated by reference herein in its entirety.

In some embodiments, a nucleic acid targeting agent is an oligonucleotide configured to bind to SV2A mRNA. In some embodiments, the oligonucleotide is at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 99.1%, 99.2%, 99.3%. 99.4%, 99.5%, 99.6%, 99.7%, 99.8%, 99.9%, or 100% identical or complementary to a region of SEQ ID NO:19. In some embodiments, the region is a region having, having at least, or having at most 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides, or any range derivable therein, starting at any position of SEQ ID NO:19. In some embodiments, a nucleic acid targeting agent is an oligonucleotide configured to bind to SV2B mRNA. In some embodiments, the oligonucleotide is at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 99.1%, 99.2%, 99.3%, 99.4%, 99.5%, 99.6%, 99.7%, 99.8%, 99.9%, or 100% identical or complementary to a region of SEQ ID NO:21. In some embodiments, the region is a region having, having at least, or having at most 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides, or any range derivable therein, starting at any position of SEQ ID NO:21. In some embodiments, a nucleic acid targeting agent is an oligonucleotide configured to bind to SV2A mRNA. In some embodiments, the oligonucleotide is at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 99.1%, 99.2%, 99.3%, 99.4%, 99.5%, 99.6%, 99.7%, 99.8%, 99.9%. or 100% identical or complementary to a region of SEQ ID NO:23. In some embodiments, the region is a region having, having at least, or having at most 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides, or any range derivable therein, starting at any position of SEQ ID NO:23.

In some embodiments, an SV2 protein-targeting agent is an SV2 protein-binding agent. An SV2 protein-binding agent describes a compound capable of binding to and mediating degradation of an SV2 protein. An SV2 protein-binding agent may be an SV2A-binding agent. An SV2 protein-binding agent may be an SV2B-binding agent. An SV2 protein-binding agent may be an SV2C-binding agent. In some embodiments, an SV2 protein-binding agent of the disclosure is levetiracetam ((S)-(−)-α-ethyl-2-oxo-1-pyrrolidine acetamide; also “ucb L059”) or a derivative thereof. Levetiracetam is described in, for example, U.S. Pat. No. 4,943,639 and Gower AJ, et al. Eur J Pharmacol. 1992 Nov. 10;222 (2-3): 193-203, each incorporated by reference herein in their entirety. As disclosed herein, under certain conditions, levetiracetam is capable of stimulating degradation of an SV2 protein (e.g., SV2A) in a cell. In some embodiments, an SV2 protein-binding agent of the disclosure is brivaracetam ((2S)-2-[(4R)-2-oxo-4-propyl-pyrrolidin-1-yl]butanamide) or a derivative thereof. Brivaracetam and derivatives thereof are described in, for example, U.S. Pat. Nos. 8,034,958 and 8,563,036, each of which is incorporated herein by reference in its entirety. As disclosed herein, under certain conditions, brivaracetam is capable of stimulating degradation of an SV2 protein (e.g., SV2A) in a cell. In some embodiments, an SV2 protein-binding agent of the disclosure is padsevonil ((4R)-4-(2-chloro-2,2-difluoroethyl)-1-{[2-(methoxymethyl)-6-(trifluoromethyl)imidazo[2,1-b][1,3,4]thiadiazol-5-yl]methyl}pyrrolidin-2-one) or a derivative thereof. Padsevonil and derivatives thereof are described in, for example, U.S. Pat. No. 8,822,508, incorporated herein by reference in its entirety.

Certain aspects of the disclosure are directed to methods for reducing the amount of a cell surface receptor on a cell. Some embodiments are directed to methods for reducing the amount of a cell surface receptor by administering to a cell an agent that reduces the amount of an SV2 protein (e.g., an SV2 protein-targeting agent). As disclosed herein, reducing the amount of an SV2 protein can lead to a reduction in the amount or one or more cell surface proteins regulated by the SV2 protein. For example, aspects of the present disclosure describe regulation of the cell surface receptor ST2L by SV2A, such that reducing the amount of SV2A in a cell (e.g., by promoting SV2A degradation) results in a reduced amount of ST2L in the cell. Therefore, in one example, a method of the disclosure comprises reducing the amount of ST2L in a cell by administering an agent that reduces the amount of SV2A in the cell (e.g., an agent such as levetiracetam, brivaracetam, etc.). In some embodiments, a cell surface receptor of the disclosure is angiotensin converting enzyme 2 (ACE2). Therefore, in one example, a method of the disclosure comprises reducing the amount of ACE2 in a cell by administering an SV2 protein-targeting agent to the cell. In some embodiments, a cell surface receptor of the disclosure is programmed cell death protein 1 (PD-1). Therefore, in one example, a method of the disclosure comprises reducing the amount of PD-1 in a cell by administering an SV2 protein-targeting agent to the cell.

### II. Proteins

As used herein, a “protein” or “polypeptide” refers to a molecule comprising at least five amino acid residues. A “peptide.” as used herein, refers to a molecule comprising at least three amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

### A. SV2 Proteins

Aspects of the disclosure relate to proteins of the synaptic vesicle glycoprotein 2 (SV2) family (also “SV2 proteins”). Without wishing to be bound by theory, the SV2 family is understood to contain three SV2 proteins; SV2A, SV2B, and SV2C. Various attributes of SV2proteins have been described in the art. Certain properties of SV2 proteins, including SV2A, SV2B, and SV2C, are described in, for example, Stout, K. A., et al. (2019). ACS chemical neuroscience, 10(9),3927-3938, incorporated herein by reference in its entirety. As used herein, “SV2 mRNA” describes a messenger RNA (mRNA) that encodes for an SV2 protein. “SV2A mRNA” describes an mRNA that encodes for the SV2A protein. “SV2B mRNA” describes an mRNA that encodes for the SV2B protein. “SV2C mRNA” describes an mRNA that encodes for the SV2C protein.

### 1. SV2A

Aspects of the disclosure relate to synaptic vesicle glycoprotein 2A (SV2A). SV2A (NCBI Protein RefSeq NP_055664; SEQ ID NO:18) is a transmembrane protein encoded by the gene Sv2a (NCBI mRNA Refseq NM_014849; SEQ ID NO:19).

### 2. SV2B

Aspects of the disclosure relate to synaptic vesicle glycoprotein 2B (SV2B). SV2B (NCBI Protein RefSeq NP_055663; SEQ ID NO:20) is a transmembrane protein encoded by the gene Sv2b (NCBI mRNA Refseq NM_014848; SEQ ID NO:21).

### 3. SV2C

Aspects of the disclosure relate to synaptic vesicle glycoprotein 2C (SV2C). SV2C (NCBI Protein RefSeq NP_055794; SEQ ID NO:22) is a transmembrane protein encoded by the gene Sv2c (NCBI mRNA Refseq NM_014979; SEQ ID NO:23).

### B. ST2

Aspects of the disclosure relate to Interleukin-1 receptor-like 1, or ST2. ST2 functions as a receptor for interleukin-33 (IL-33). Without wishing to be bound by theory, there are understood to be at least two isoforms of ST2; a full-length functional ST2 (ST2L) and a soluble ST2 (sST2) that lacks the carboxyl-terminal cytoplasmic region. ST2L (NCBI Protein RefSeq NP_057316) is encoded by the gene Il1rl1 (NCBI mRNA Refseq NM_016232). As used herein, “ST2” refers to the full length functional ST2 (i.e., ST2L) unless otherwise indicated.

### C. ACE2

Aspects of the disclosure relate to angiotensin converting enzyme 2 (ACE2). ACE2 functions to convert angiotensin I to angiotensin 1-9 and angiotensin II to angiotensin 1-7. ACE2 serves as a receptor for and to facilitate entry of human coronaviruses SARS-COV and SARS-CoV-2, as well as human coronavirus NL63/HCoV-NL63. ACE2 (NCBI Protein RefSeq NP_068576) is encoded by the gene ACE2 (NCBI mRNA Refseq NM_021804).

### D. PD-1

Aspects of the disclosure relate to programmed cell death protein 1 (PD-1). PD-1m functions as an inhibitory receptor on antigen activated T-cells. The PDCD1-mediated inhibitory pathway is exploited by tumors to attenuate anti-tumor immunity and escape destruction by the immune system, thereby facilitating tumor survival. PD-1 (NCBI Protein RefSeq NP_005009) is encoded by the gene PDCDI (NCBI mRNA Refseq NM_005018).

### III. Group 2 Innate Lymphoid Cells (ILC2s) and Associated Conditions

Aspects of the present disclosure relate to group 2 innate lymphoid cells (ILC2s). Without wishing to be bound by theory, ILC2s are understood to be associated with (i.e., involved in the pathology of) various conditions including certain immune disorders and cancer. As used herein, an “ILC2-associated condition,” or “condition associated with ILC2s” describes a condition in which ILC2s play a role in the development, maintenance, and/or progression of the condition. ILC2s and certain ILC2-associated conditions are described in, for example, Barlow JL, Mckenzie ANJ. Annu Rev Physiol. 2019 Feb. 10;81:429-452 and Moral JA, et al. Nature. 2020 March;579 (7797): 130-135, each incorporated herein by reference in their entirety.

Examples of conditions associated with ILC2s include autoimmune conditions, inflammatory conditions, allergic disorders, obesity, pneumonia (including, e.g., COVID-19 pneumonia) and cancer. In some embodiments, the condition associated with ILC2s is an autoimmune condition. In some embodiments, the condition associated with ILC2s is an allergic disorder. In some embodiments, the condition associated with ILC2s is obesity. In some embodiments, the condition associated with ILC2s is pneumonia, which may be viral pneumonia such as COVID-19 pneumonia. In some embodiments, the condition associated with ILC2s is cancer.

In some embodiments, the condition associated with ILC2s is pneumonia. In some embodiments, disclosed are methods for treating a subject having one or more symptoms of pneumonia. Symptoms of pneumonia are recognized in the art and include, for example, cough, fever, chills, shortness of breath, chest pain, nausea and vomiting, and fatigue. In some embodiments, a subject has been diagnosed with pneumonia. In some embodiments, a subject is suspected of having pneumonia. In some embodiments, the pneumonia is viral pneumonia. In some embodiments, the pneumonia is COVID-19 pneumonia (i.e., pneumonia resulting from a SARS-CoV-2 infection).

In some embodiments, the condition associated with ILC2s is an autoimmune or inflammatory condition. The autoimmune condition or inflammatory condition amenable for treatment by methods and compositions of the disclosure (e.g., SV2 protein-targeting agents such as levetiracetam and brivaracetam) may include, but not be limited to conditions such as diabetes (e.g. Type 1 diabetes), graft rejection, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, Type II collagen-induced arthritis, infectious arthritis, Lyme arthritis, proliferative arthritis, psoriatic arthritis, Still's disease, vertebral arthritis, and systemic juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, X-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, transmural colitis, and/or autoimmune inflammatory bowel disease), pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano-or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, cythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus.

### IV. Viruses

Aspects of the current disclosure include methods and compositions related to one or more viruses, or components (e.g., proteins, nucleic acid) thereof or derived therefrom, including methods and compositions for treatment or prevention of a viral infection. In some embodiments, the virus is a DNA virus. In some embodiments, the virus is an RNA virus. Various viruses are recognized by those skilled in the art and are contemplated herein.

### A. Coronaviruses

In particular embodiments, the virus is from the family Coronaviridae. Coronaviridae is a family of enveloped, positive-sense, single-stranded RNA viruses. Coronavirus is the common name for Coronaviridae and Orthocoronavirinae (also referred to as Coronavirinae). The family Coronaviridae is organized in 2 sub-families, 5 genera, 23 sub-genera and about 40 species. They are enveloped viruses having a positive-sense single-stranded RNA genome and a nucleocapsid having helical symmetry. The genome size of coronaviruses ranges from about 26-32 kilobases.

The present disclosure encompasses treatment or prevention of infection of any virus in the Coronaviridae family. In certain embodiments, the disclosure encompasses treatment or prevention of infection of any virus in the subfamily Coronavirinae and including the four genera, Alpha-, Beta-, Gamma-, and Deltacoronavirus. In specific embodiments, the disclosure encompasses treatment or prevention of infection of any virus in the genus of Betacoronavirus, including the subgenus Sarbecovirus and including the species of severe acute respiratory syndrome-related coronavirus. In specific embodiments, the disclosure encompasses treatment or prevention of infection of any virus in the species of severe acute respiratory syndrome-related coronavirus, including the strains severe acute respiratory syndrome coronavirus (SARS-COV) and severe acute respiratory syndrome coronavirus 2 (SARS-COV-2, the virus that causes COVID-19). The disclosure encompasses treatment or prevention of infection any isolate, strain, type (including Type A, Type B and Type C; Forster et al., 2020, PNAS, available on the World Wide Web at doi.org/10.1073/pnas.2004999117), cluster, or sub-cluster of the species of severe acute respiratory syndrome-related coronavirus, including at least SARS-COV-2. In specific embodiments, the virus has a genome length between about 29000 to about 30000, between about 29100 and 29900, between about 29200 and 29900, between about 29300 and 29900, between about 29400 and 29900, between about 29500 and 29900, between about 29600 and 29900, between about 29700 and 29900, between about 29800 and 29900, or between about 29780 and 29900 base pairs in length.

Examples of specific SARS-COV-2 viruses include the following listed in the NCBI GenBank® Database, and these GenBank® Accession sequences are incorporated by reference herein in their entirety: (a) LC534419 and LC534418 and LC528233 and LC529905 (examples of different strains from Japan); (b) MT281577 and MT226610 and NC_045512 and MN996531 and MN908947 (examples of different strains from China); (c) MT281530 (Iran); (d) MT126808(Brazil); (c) MT020781 (Finland); (f) MT093571 (Sweden); (g) MT263074 (Peru); (h) MT292582 and MT292581 and MT292580 and MT292579 (examples of different strains from Spain); (i) examples from the United States, such as MT276331 (TX); MT276330 (FL); MT276328 (OR) MT276327 (GA); MT276325 (WA); MT276324 (CA); MT276323 (RI); MT188341 (MN); and (j) MT276598 (Israel). In particular embodiments, the disclosure encompasses treatment or prevention of infection of any of these or similar viruses, including viruses whose genome has at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. In particular embodiments, the disclosure encompasses treatment or prevention of infection of any of these or similar viruses, including viruses whose genome has its entire sequence that is greater than 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. As one specific example, the present disclosure includes methods of treatment or prevention of infection of a virus having a genome sequence of SEQ ID NO: 1 (represented by GenBank® Accession No. NC_045512; origin Wuhan, China) and any virus having a genome sequence with at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to SEQ ID NO:1.

### V. Cancer Therapy

In some embodiments, the method further comprises administering a cancer therapy to the patient. The cancer therapy may be chosen based on the expression level measurements, alone or in combination with the clinical risk score calculated for the patient. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be an immune checkpoint therapy. Any of these cancer therapies may also be excluded. Combinations of these therapies may also be administered.

The term “cancer,” as used herein, may be used to describe a solid tumor, metastatic cancer, or non-metastatic cancer. In certain embodiments, the cancer may originate in the bladder. blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some embodiments, the cancer is recurrent cancer. In some embodiments, the cancer is Stage I cancer. In some embodiments, the cancer is Stage II cancer. In some embodiments, the cancer is Stage III cancer. In some embodiments, the cancer is Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pincaloma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; cosinophilic leukemia; monocytic leukemia; mast cell leukemia; megakaryoblastic leukemia; myeloid sarcoma; and hairy cell leukemia.

In some embodiments, the cancer is a recurrent cancer. In some embodiments, the cancer is Stage I cancer. In some embodiments, the cancer is Stage II cancer. In some embodiments, the cancer is Stage III cancer. In some embodiments, the cancer is Stage IV cancer.

It is contemplated that a cancer treatment may exclude any of the cancer treatments described herein. Furthermore, embodiments of the disclosure include patients that have been previously treated with a therapy described herein, are currently being treated with a therapy described herein, or have not been treated with a therapy described herein. In some embodiments, the patient is one that has been determined to be resistant to a therapy described herein. In some embodiments, the patient is one that has been determined to be sensitive to a therapy described herein.

### VI. Administration of Therapeutic Compositions

The therapy provided herein may comprise administration of a combination of therapeutic agents, such as a first cancer therapy (e.g., an SV2 protein-targeting agent as described herein, such as levetiracetam) and a second cancer therapy (e.g., a chemotherapy, an immunotherapy, etc.). The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the therapeutic compositions may be employed.

The therapeutic agents of the disclosure may be administered by the same route of administration or by different routes of administration. In some embodiments, the first cancer therapy is administered intravenously, intramuscularly, subcutancously, topically, orally, transdermally, intraperitoneally, intraorbitally, by implantation, by inhalation, intrathecally, intraventricularly, or intranasally. In some embodiments, the second cancer therapy is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose (or “effective amount”) is understood to refer to an amount necessary to achieve a particular effect (e.g., treatment of a condition, prevention of a condition, alleviation of symptoms associated with a condition, etc.). In the practice in certain embodiments, it is contemplated that doses in the range from 10 mg/kg to 200 mg/kg can affect the protective capability of these agents. Thus, it is contemplated that doses include doses of about 0.1. 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one that can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

In some embodiments, the disclosure comprises administering to a subject an effective amount of levetiracetam. In some embodiments, levetiracetam is administered to the subject as an oral composition. In such embodiments, a daily dosage may be between 1 mg and 2000 mg, between 1 mg and 1000 mg, or between 1 mg and 500 mg. In some embodiments, levetiracetam is administered to a subject at a dose of at least, at most, or about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 mg per day, or any range or value derivable therein. In some embodiments, levetiracetam is administered to a subject at a dose of about 250 mg per day. In some embodiments, levetiracetam is administered to a subject at a dose of about 500 mg per day. In some embodiments, levetiracetam is administered to a subject at a dose of about 750 mg per day. In some embodiments, levetiracetam is administered to a subject at a dose of about 1000 mg per day.

In some embodiments, the disclosure comprises administering to a subject an effective amount of brivaracetam. In some embodiments, brivaracetam is administered to the subject as an oral composition. In some embodiments, brivaracetam is administered to the subject as a composition formulated for intravenous injection. In such embodiments, a daily dosage may be between 1 mg and 2000 mg, between 1 mg and 1000 mg, or between 1 mg and 500 mg. In some embodiments, brivaracetam is administered to a subject at a dose of at least, at most, or about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 700, 2800, 2900, 3000, 3100, 3200, 3300, 3400, 3500, 3600, 3700, 3800, 3900, or 4000 mg per day, or any range or value derivable therein. In some embodiments, brivaracetam is administered to a subject at a dose of about 50 mg per day. In some embodiments, brivaracetam is administered to a subject at a dose of about 100 mg per day. In some embodiments, brivaracetam is administered to a subject at a dose of about 150 mg per day. In some embodiments, brivaracetam is administered to a subject at a dose of about 200 mg per day. In some embodiments, brivaracetam is administered to a subject at a dose of at least, at most, or about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mg/kg per day.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VII. General Pharmaceutical Compositions

In some embodiments, pharmaceutical compositions are administered to a subject. Different aspects may involve administering an effective amount of a composition to a subject. In some embodiments, an agent capable of reducing the amount of an SV2 protein (e.g., an SV2protein-targeting agent such as levetiracetam or brivaracetam, an antisense oligonucleotide capable of reducing SV2 protein expression, etc.) is administered to a subject. Additionally, such compositions can be administered in combination with an additional therapeutic agent (e.g., a chemotherapeutic, an immunotherapeutic). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaccous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle, which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are specific to each individual. Factors affecting dose include physical and clinical state of the subject, route of administration, intended goal of treatment (alleviation of symptoms versus cure), and potency, stability, and toxicity of the particular composition.

### Examples

The following examples are included to demonstrate certain embodiments of the disclosure. It should be appreciated by those of skill in the art that the techniques disclosed in the examples which follow represent techniques discovered to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Investigation of the Role of SV2A in IL-33 Signaling and Lung Inflammation

### Methods

### Mice and Treatments

Sv2a^{+/-} mice on C57BL/6 129Sv mixed genetic background²¹ were purchased from the Jackson laboratory (B6;129P2-Sv2a^{tm1Sud}Sv2b^{tm1Sud}/J) and were backcrossed to C57 background for at least 5 generations prior to use. Mice were housed under specific-pathogen-free conditions. Sex-matched littermates between 6 and 16 weeks of age were used for experiments if not otherwise indicated. C57BL/6 mice were purchased from the Jackson Laboratory or the UCLA animal core facility. C57BL/6 mice were randomly assigned to treatment groups after matching for sex and age. Mice were anaesthetized with isoflurane and treated intranasally with the indicated stimuli [100 ng IL-33 (R&D), 17 mg/kg LEV (MedKoo Biosciences), or 50 μg papain (Sigma)] for either 4 hrs or daily for three consecutive days (3D) where indicated. For the IL-33 or papain-induced lung inflammation models, mice were treated intranasally on days 1, 2, and 3. Bone marrows and lungs were harvested, processed, and analyzed on day 4. The inventors were not blinded to allocation during the studies. All studies were conducted in accordance with animal protocols approved by the Animal Research Committee of the University of California Los Angeles.

### Primary Cell Cultures

Primary mouse embryonic fibroblast cells (MEFs) were prepared as previously described³⁵. Briefly, mouse embryos were collected around embryonic day 14. Visible organs such as the brain, liver, and spleen were removed, and the leftover tissue was quickly minced and incubated in 0.25% trypsin-EDTA for 2 hr at 37° C. Cells were then washed with media and seeded in DMEM with 10% FBS, 1% penicillin/streptomycin and 0.055 mM β-mercaptoethanol. Every three days cells were passaged. Starting at passage 2, cells were used for studies.

### Flow Cytometry Analysis

Mice were euthanized and perfused with cold PBS. Lung lobes were diced into small pieces and incubated with 0.2 mg/mL collagenase P. 0.8 mg/mL Dispase II, 0.1 mg/mL DNase I in RPMI1640 containing 5% FBS for 1 hr at 37° C. before being mashed through 70-mm cell strainers. Bone marrow cells were collected by flushing out the marrow from femurs and tibias using a syringe with PBS. Red blood cells were removed using RBC lysis buffer (Sigma-Aldrich, Cat #: R7757) according to the manufacturer's recommendations followed by 1×PBS wash. After saturating the Fc-receptors with CD16/CD32-blocking antibody (BioLegend), single-cell suspensions were incubated on ice with conjugated antibodies in PBS (Ca²⁺ and Mg²⁺-free).

Dead cells were routinely excluded with Zombie UV Fixable Viability Kit (BioLegend) or Fixable Aqua Dead Cell Stain (Thermo Fisher Scientific). Lineage-positive cells were excluded by staining for B220 (RA3-6B2), CD38 (145-2C11), TCRβ (H57-597), TCRγδ (eBioGL3), Ter119 (Ter119), NK1.1 (PK136), CD11b (M1/70), and CD11c (N418). For surface staining CD45 (30-F11), KLRG1 (2F1), CD25 (PC61.5), CD127 (A7R34), Sca1 (D7), Thy1/CD90.2 (53-2.1), and ST2 (RMST2-2, eBioscience) were used. For intracellular staining, cells were fixed and permeabilized with the Transcription Buffer Set (BD Pharmingen) per manufacturer's instructions followed by staining with anti-GATA3 (TWAJ, eBioscience), anti-IL-5 (TRFK5, Biolegend) and anti-IL-13 (eBio13A, eBioscience). Stained cells were used for flow cytometry analysis using a LSRII or Attune NxT (Becton Dickinson, BD) flow cytometer. Data were analyzed using the FlowJo software. Different cell types were identified by the following gating strategies^{36,37}: lung ILC2s (CD45⁺Lin KLRG1⁺CD90⁺ or CD45⁺Lin GATA3⁺CD90⁺CD25⁺), BM ILC2P (CD45⁺Lin⁻ CD127⁺CD25⁺).

### Quantitative Real-Time Polymerase Chain Reaction (Q-PCR) Analysis

Quantitative real time polymerase chain reaction (Q-PCR) analyses were performed as described³⁸. Briefly, cDNA prepared using the iScript cDNA Synthesis kit (Bio-Rad) were analyzed by Q-PCR using the CFX-96 Real-Time Detection System (Bio-Rad). The Q-PCR primer sequences of genes were: Sv2a forward—TGGATGATCGGTGGAGTGTA (SEQ ID NO:2), Sv2a reverse CACACACGAGGACAAACACC (SEQ ID NO:3), St2l forward AAGGCACACCATAAGGCTGA (SEQ ID NO:4), St2l reverse TCGTAGAGCTTGCCATCGTT (SEQ ID NO:5), sSt2 forward—TCGAAATGAAAGTTCCAGCA (SEQ ID NO:6), sSt2 reverse TGTGTGAGGGACACTCCTTAC (SEQ ID NO:7). Il1rap forward—CCCACCCTCCTCAATGACAC (SEQ ID NO:8), Il1rap reverse—GCACTGGGAATCTCATGGCA (SEQ ID NO:9), Il5 forward—AGCACAGTGGTGAAAGAGACCTT (SEQ ID NO:10), Il5 reverse—TCCAATGCATAGCTGGTGATTT (SEQ ID NO:11), Il13 forward—CCTGGCTCTTGCTTGCCTT (SEQ ID NO:12), Il13 reverse—GGTCTTGTGTGATGTTGCTCA (SEQ ID NO:13), Hprt forward—CAGTACAGCCCCAAAATGGT (SEQ ID NO:14), Hprt reverse—CAAGGGCATATCCAACAACA (SEQ ID NO:15), Gapdh forward—TGTTCCTACCCCCAATGTGT (SEQ ID NO:16), Gapdh reverse—GGTCCTCAGTGTAGCCCAAG (SEQ ID NO:17).

### Immunoblot Analysis

Protein extracts from cells or tissues were prepared using whole protein lysis buffer (WL buffer) containing 50 mM Tris (pH 8), 0.4M NaCl, 0.5% NP-40, 10% glycerol, 1 mM EDTA, 1 mM DTT, and 1 mM PMSF as described previously³⁹. To extract proteins from MEFs, cells were washed with PBS once and then lysed with WL buffer on ice for 30 min. Lung or brain tissues from mice that had been euthanized and perfused with cold PBS were directly homogenized in the presence of WL buffer or alternatively, tissues were briefly disrupted in PBS and the insoluble fractions were then lysed in WL buffer for protein extraction. Immunoblot analysis was performed using the following antibodies: anti-ST2L (R3880-2, Abiocode), anti-ST2 (M3880-1, Abiocode), anti-SV2A (M3004-1, Abiocode), anti-Tubulin (M0267-1b, Abiocode).

### Cell-Free Protein Stability Assay

Proteins extracts were prepared by incubating WL buffer with brain or lung tissues that had been pre-washed with PBS. Reaction mixtures were set up by adding 12 μl of protein extracts (1-5 μg/μl) with 8 μμl of ddH20 or 8 μl of various concentrations of LEV (prepared by dissolving in ddH2O). The reaction mixtures were subsequently incubated at 37° C. for 1 hr, and the reactions were terminated by adding 20 μl of 4×SDS sample buffer followed by denaturing at 95° C. for 5 min. The samples were then analyzed directly by immunoblot analysis.

### Results

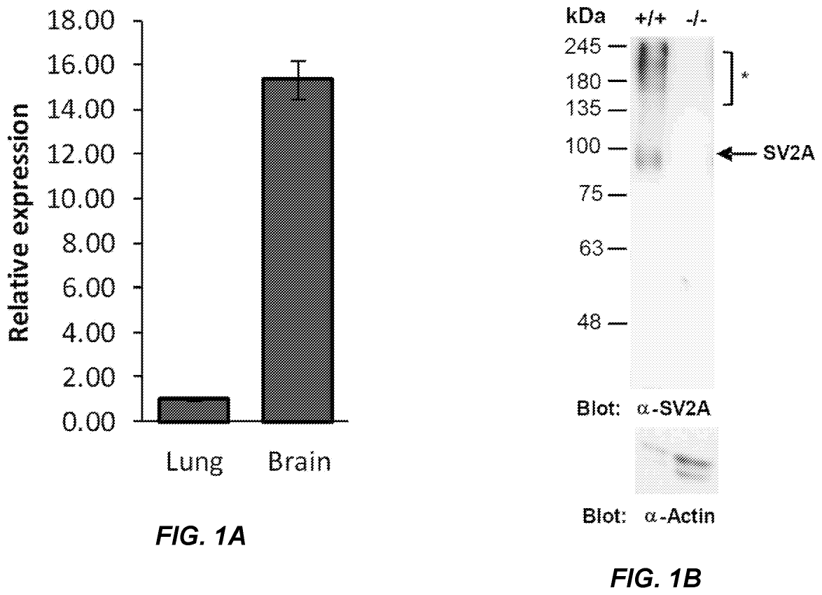

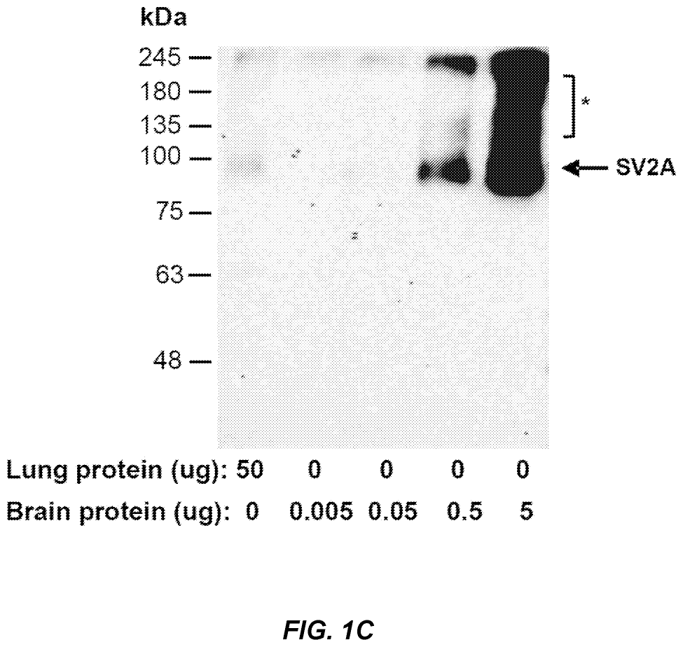

SV2A, a member of the synaptic vesicle glycoprotein 2 (SV2) family found in vesicles of neuronal or endocrine cells, plays a key role in epilepsy pathophysiology. Levetiracetam (LEV) is a FDA-approved small molecule anti-seizure drug that targets SV2A¹⁶⁻¹⁸. Despite extensive research effort, the molecular basis of SV2A function and the mechanistic effect of LEV binding remains be unclear¹⁶⁻¹⁸. To explore the potential role of SV2A in the peripheral system, the expression of SV2A was examined in mouse lungs. The levels of SV2A mRNA and protein in the lung were about 10-fold lower or at least 100-fold lower as compared to the levels in the brain, respectively (FIGS. 1A-1C).

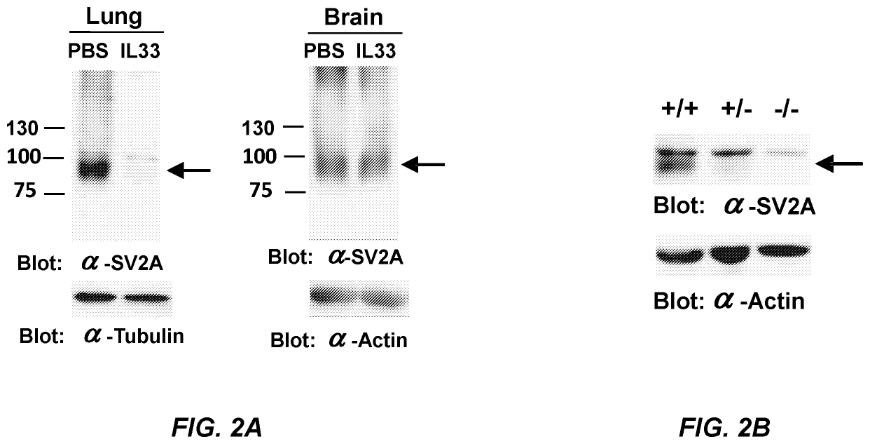

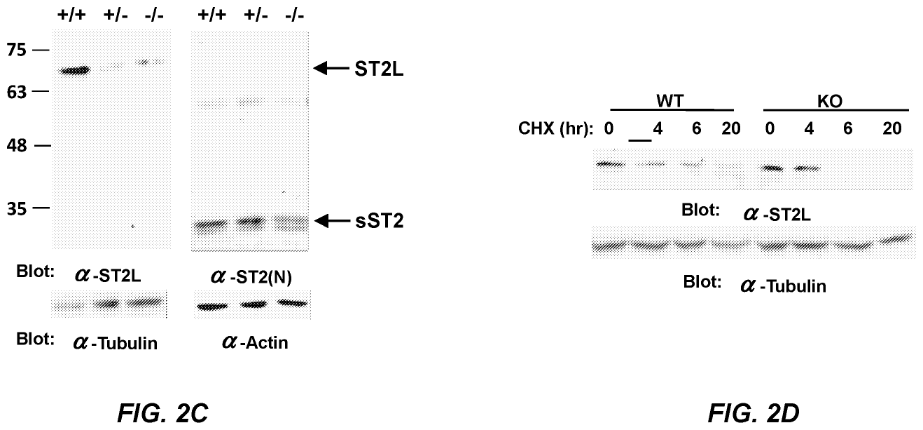

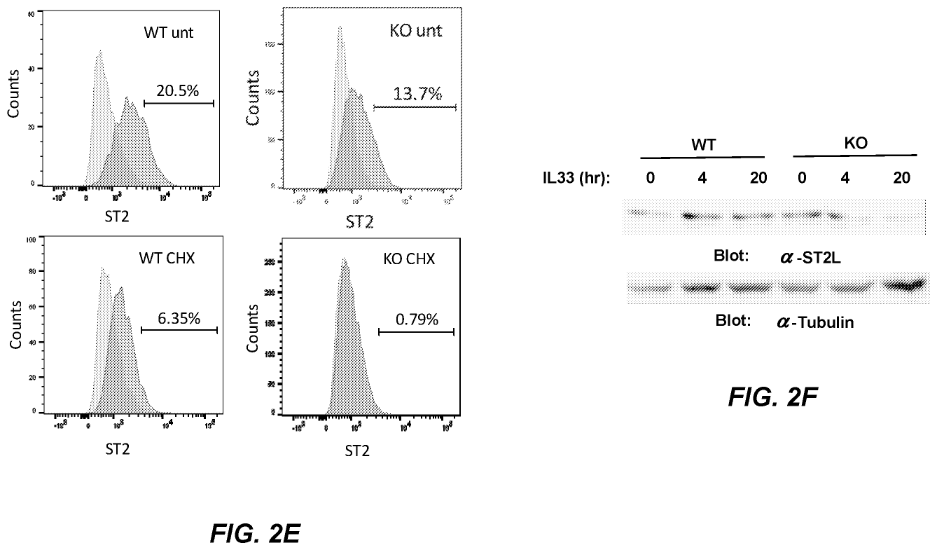

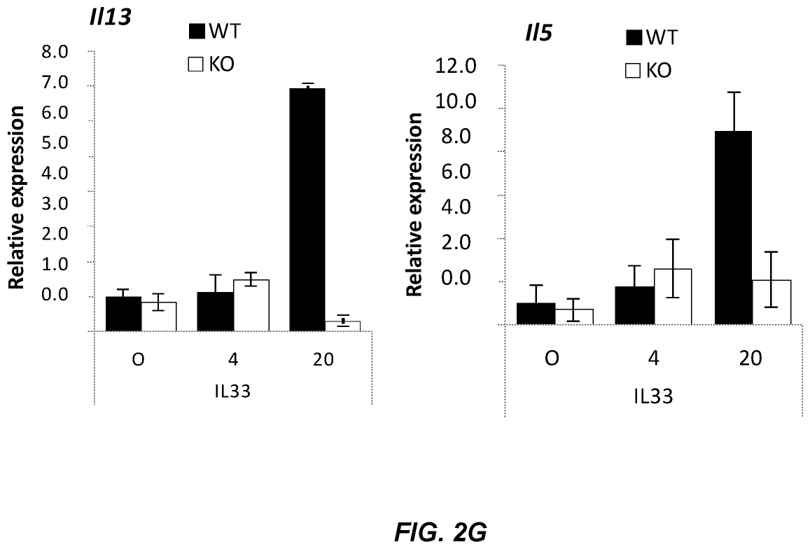

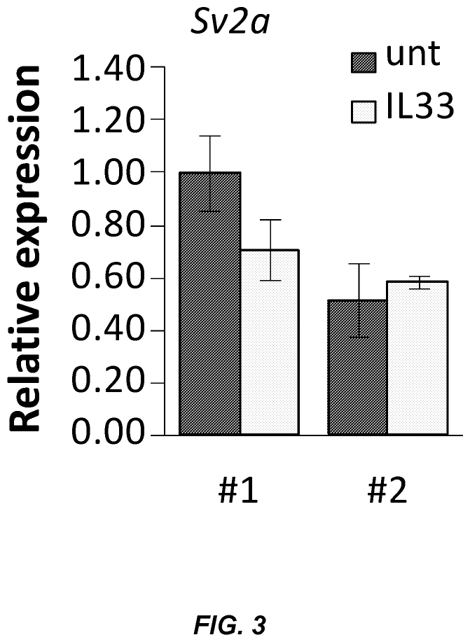

IL-33 is an alarmin cytokine that plays a key role in both the peripheral and central nervous system (CNS) immune responses^{7,9,11,19}. Intranasal administration of IL-33 caused the reduction of SV2A protein in the lungs, but not in the brain where SV2A is predominantly expressed in neuronal cells that express low levels of IL-33 receptor²⁰ (FIG. 2A). The IL-33-triggered reduction of SV2A protein occurs at the post-transcriptional level since IL-33 treatment did not suppress the levels of Sv2a mRNA (FIG. 3). Previous studies showed that Sv2a^{-/-} mice die within 2-3 weeks after birth while Sv2a^{+/-} mice are normal in development^{21,22}. Protein extracts were prepared from the lungs of Sv2a^{+/+}, Sv2a^{+/-}, and Sv2a^{-/-} littermate pups at the age of 12 days, followed by immunoblot analysis with anti-SV2A. SV2A protein was dramatically reduced in the lungs of Sv2a^{+/-} pups and was completely absent in the lungs of Sv2a^{-/-} pup (FIG. 2B).

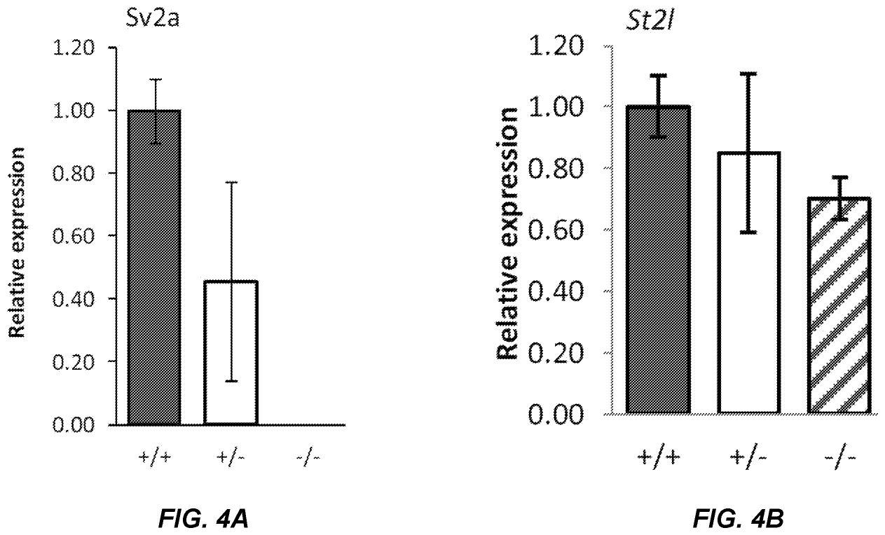

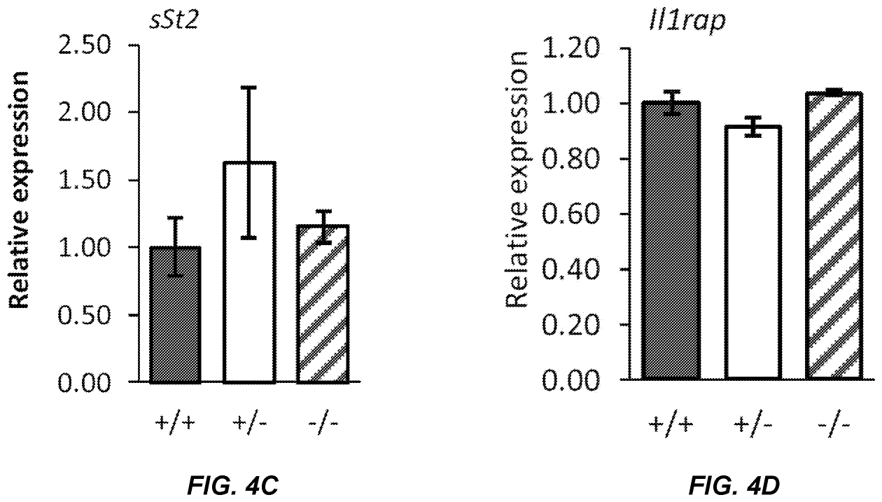

There are at least two isoforms of the IL-33 receptor: the full-length functional ST2(ST2L) and the soluble ST2 (sST2) that lacks the carboxyl-terminal cytoplasmic region^{7,9}. Immunoblot analysis was performed with a polyclonal antibody (anti-ST2L) that specifically recognizes ST2L, but not sST2. Two major forms of ST2L were reported to be present on the cell surface: the 70 kD glycosylated form and the 60 kD non-glycosylated form²³. The 70 kD glycosylated ST2L was predominantly present in the lungs and was clearly reduced in Sv2a^{+/-} and Sv2a^{-/-} pups as compared to their wild type (WT) control (FIG. 2C). Immunoblot analysis with a monoclonal anti-ST2 antibody that recognizes the N-terminal region of ST2 showed a modest reduction of sST2 protein in Sv2a^{-/-} pups (FIG. 2C). This monoclonal anti-ST2 was unable to recognize the glycosylated ST2L due to the interference of glycosylation, a phenomenon that had been reported before²⁴. ST2L and sST2 are derived from alternative RNA splicing from the same gene Il1r1²⁵⁻²⁷. Q-PCR analysis showed that Sv2a disruption in the lungs of Sv2a^{-/-} pups caused a modest reduction of St2l mRNA (FIG. 4B), but had no significant effect on the levels of sSt2(FIG. 4C) nor Il1rap (FIG. 4D), the gene that that encodes the IL-33 co-receptor IL1rap.

Next, the inventors tested whether SV2A affects protein stability of ST2L in a cell culture system. WT and Sv2a knockout primary embryonic fibroblasts (MEFs) were treated with the protein synthesis inhibitor cycloheximde (CHX) to allow the assessment of ST2L protein stability in the absence of new protein synthesis. In WT MEFs, ST2L expression was only modestly reduced 6 hr after CHX treatment, and remained to be detectable in cells treated with CHX for 20 hr. In contrast, ST2L was no longer detectable 6 hr after CHX treatment in Sv2a KO MEFs (FIG. 2D). Thus, ST2L protein was less stable in the absence of SV2A, suggesting that SV2A is required for the stabilization of ST2L protein.

To further validate these results, flow cytometry analysis of WT and Sv2a KO MEFs was performed with an independent ST2 antibody to examine the cell surface expression of ST2L (FIG. 2E). In WT primary MEFs, CHX treatment for 20 hr caused about 3-fold reduction of ST2⁺ cells (from 20.5% to 6.35%). In contrast, the percentage of ST2+ cells was suppressed by more than 15-fold in Sv2a KO MEFs treated with CHX for the same time (from 13.7% to 0.78%). These results indicated that SV2A is indeed required for the stable expression of ST2L on the cell surface.

Next, the inventors tested whether SV2A is important for ST2L expression in response to IL-33 stimulation. In WT MEFs, the level of ST2L was not reduced by IL-33. In contrast, treatment of Sv2a KO MEFs with IL-33 for 20 hr caused a dramatic reduction of ST2L, consistent with an important role of SV2A in the stabilization of ST2L protein (FIG. 2F). To further validate the important role of SV2A in IL-33 signaling, the induction of Il13 and Il5, two signature IL-33-target genes, in WT and Sv2a KO MEFs was examined. The IL33-triggered induction of Il13 and Il5 were significantly increased in WT MEFs, but not Sv2a^{-/-} (KO) MEFs (FIG. 2G). These results supported the conclusion that SV2A is crucial for maintaining the level of ST2L expression and the IL-33-induced gene activation.

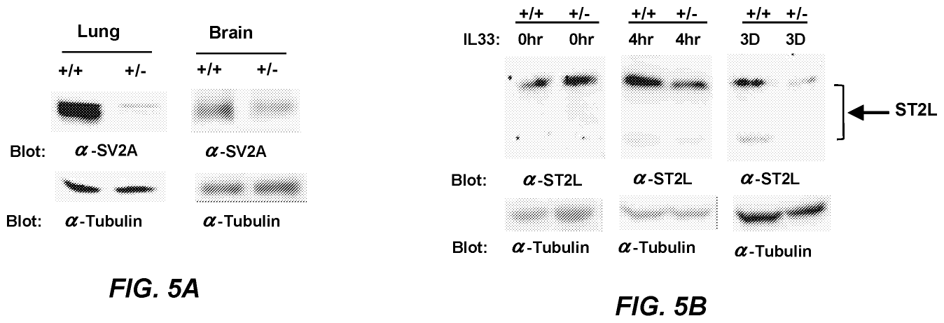

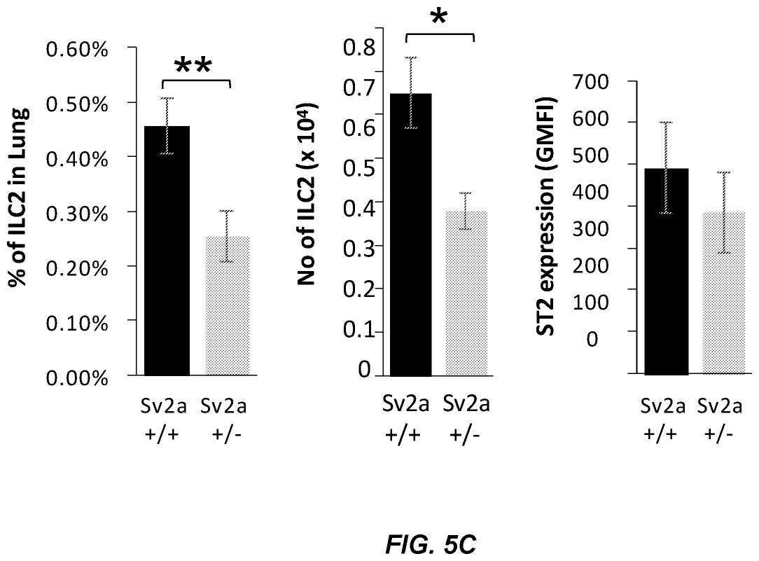

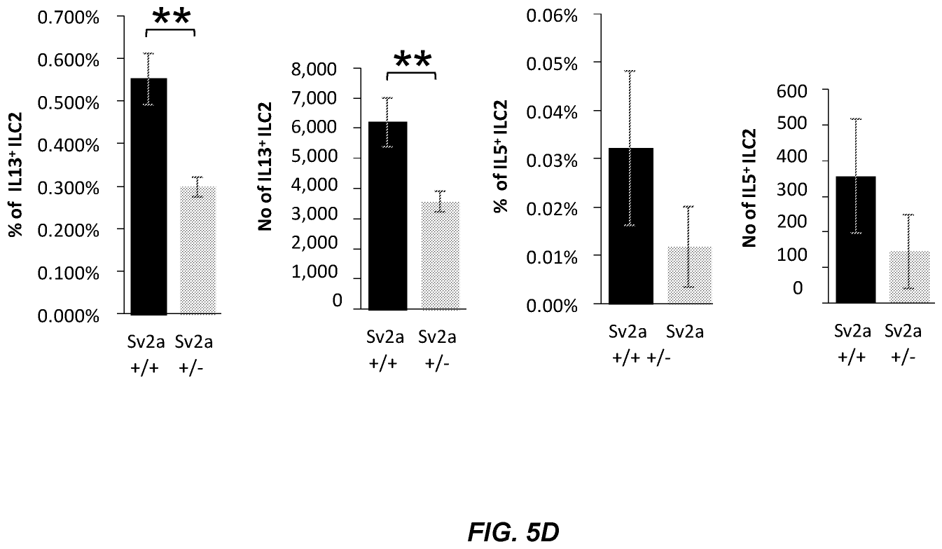

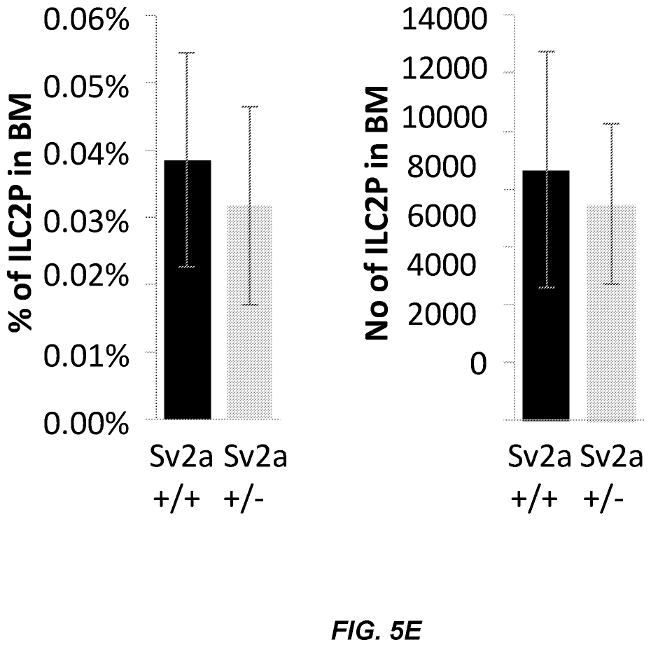

Next, the role of SV2A in IL-33 signaling was examined using Sv2a gene-targeting animal models. Immunoblot analysis showed that Sv2a heterozygosity caused a more dramatic reduction of SV2A protein in the lungs as compared to the brain of Sv2a^{+/-} adult mice (FIG. 5A). Unlike the results from the Sv2a^{+/-} pups, no significant reduction of ST2L protein in the lungs of young adult Sv2a^{+/-} mice was detected. During mouse development, the levels of IL-33 in the lungs peak during postnatal days 7-10, and become gradually reduced toward the adulthood²⁸⁻³⁰. The level of ST2L was significantly repressed upon IL-33 treatment in MEFs (FIG. 2F). The SV2A-mediated regulation of ST2L may be more evident upon IL-33 stimulation. Thus, WT and Sv2a^{+/-} mice were challenged with IL-33 for 4 hr or daily for three consecutive days (3D). The reduction of ST2L was clearly observed in Sv2a^{+/-} mice as compared to the WT control upon IL-33 stimulation (FIG. 5B). Taken together, these studies support an important role of SV2A in the control of ST2L expression during IL-33 signaling.

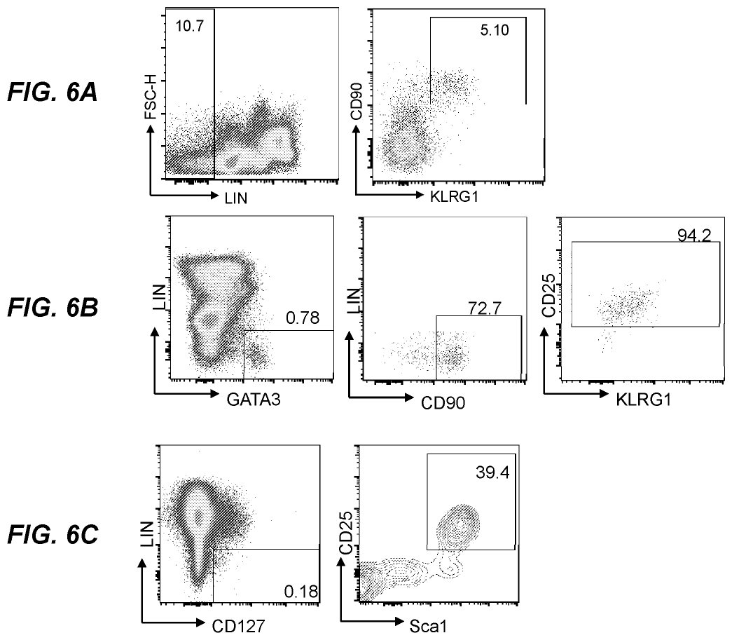

IL-33 plays a key role in the development of group 2 innate lymphoid cells (ILC2s), and systematic administration of IL-33 induces the proliferation of ILC2s^{31,32}. The inventors examined the role of SV2A in the regulation of ILC2s using the IL33-induced lung inflammation model. WT and Sv2a+/− mice were challenged with IL33 daily for three consecutive days followed by flow cytometry analysis on day 4. Sv2a heterozygosity was sufficient to cause defective lung ILC2 responses (FIG. 5C). The frequency and cell numbers of lung ILC2s (CD45⁺KLRG1⁺CD90⁺ and lacking lineage markers; see FIGS. 6A-6C) were significantly reduced in Sv2a^{+/-} mice as compared to the WT control (FIG. 5C).

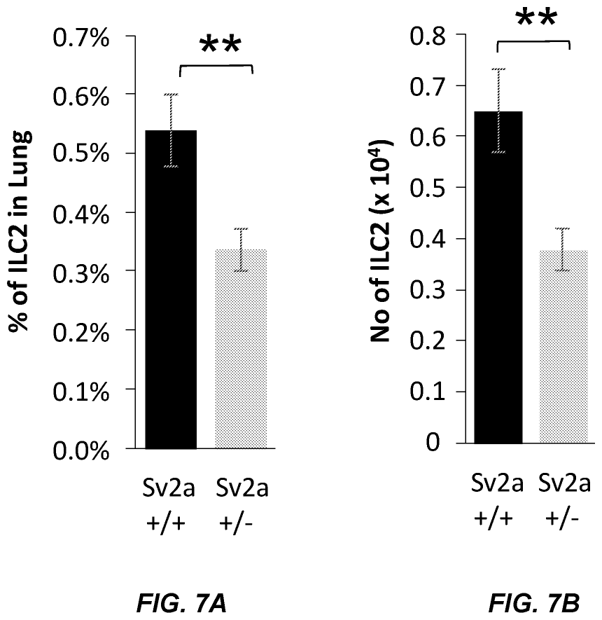

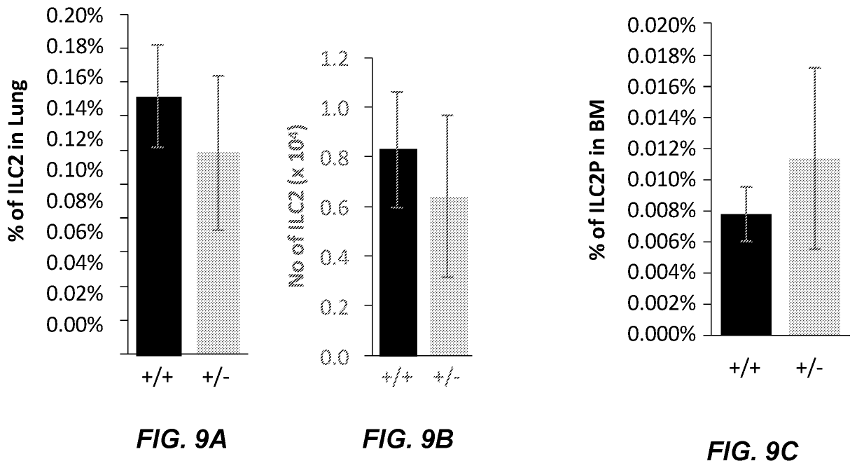

Similar results were obtained by using a different set of markers for lung ILC2s (CD45⁺GATA3⁺CD90⁺CD25⁺ and lacking lineage markers, FIGS. 7A and 7B). Furthermore, the frequency and cell numbers of IL13-producing ILC2s (IL13⁺ILC2s) were also repressed in Sv2a^{+/-} mice (FIG. 5D). These results are consistent with the role of SV2A in the regulation of ST2L expression during IL-33 signaling, and support a key physiological role of SV2A in the regulation of lung ILC2s. We also analyzed the effect of SV2A in the development of ILC2 precursor cells (ILC2P) in bone marrow (BM). No significant difference on BM ILC2P of Sv2a^{+/+} and Sv2a^{+/-} mice was observed (FIG. 5E). In addition, untreated Sv2a^{+/-} mice showed no significant defect in lung ILC2s or BM ILC2P (FIG. 9), suggesting Sv2a heterozygosity was not sufficient to affect the development of ILC2P in bone morrow or the basal levels of lung ILC2s.

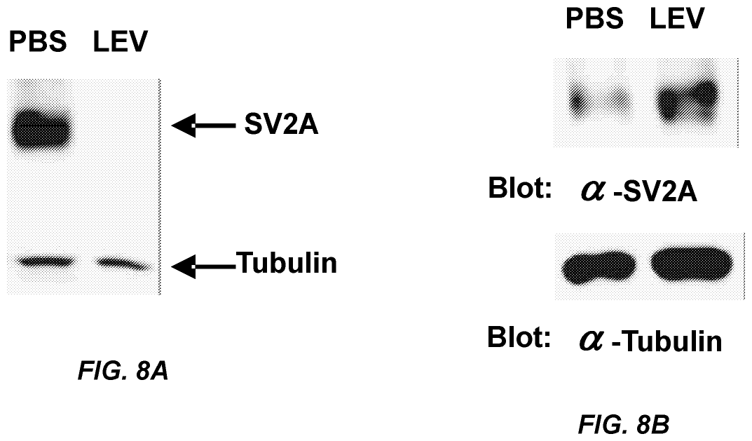

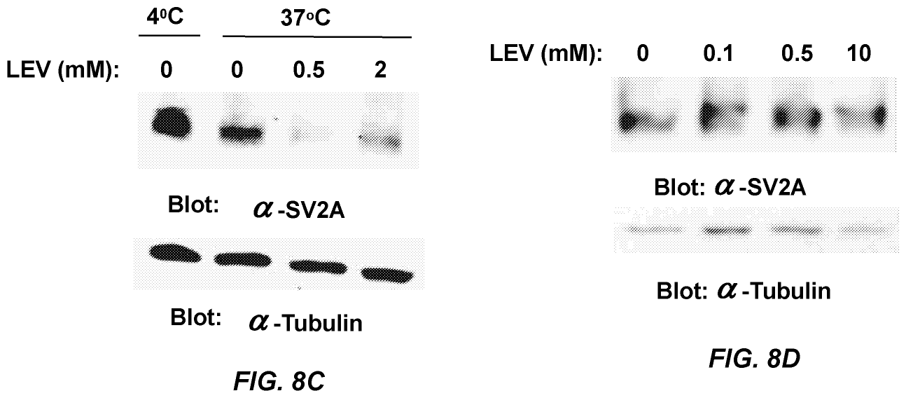

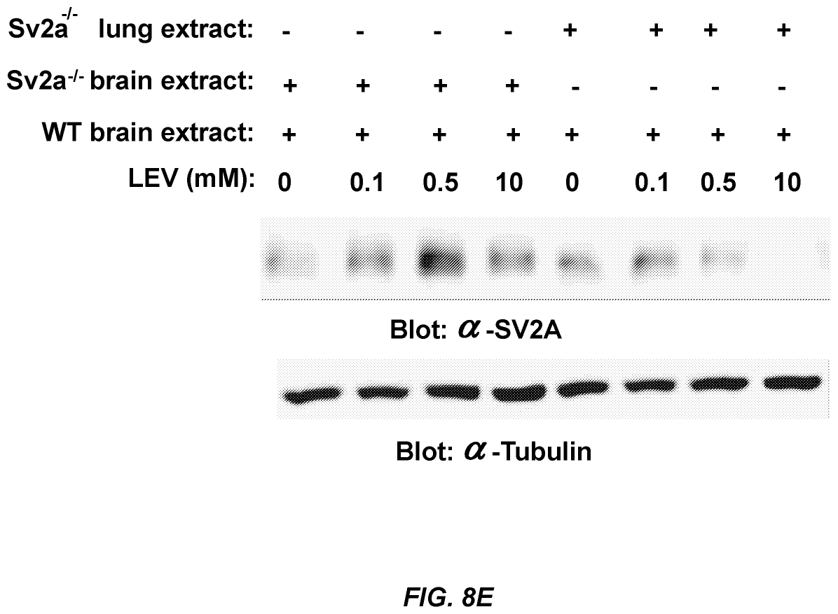

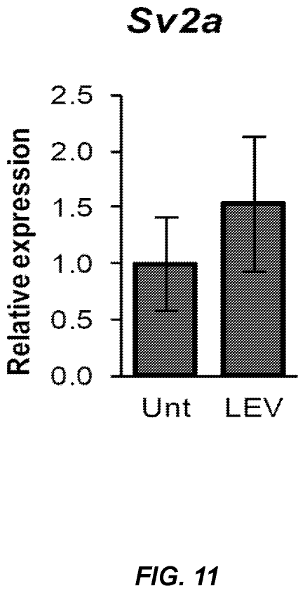

LEV targets SV2A and is a small molecule anti-seizure drug approved for the treatment of epilepsy^{18,33,34}. However, the effect of LEV on the regulation of SV2A activity is not clear. The disclosed discovery of a role of SV2A in IL-33 signaling provides a unique opportunity to analyze the molecular basis of LEV action. First, the effect of LEV on lung SV2A protein in mice treated with LEV was examined. LEV treatment for 4 hr caused a clear reduction of SV2A protein (FIG. 8A), but not Sv2a mRNA (FIG. 11) in the lungs. SV2A detected in whole brain extracts is mostly from neuronal cells. Interestingly, LEV treatment did not reduce SV2A protein in the brain (FIG. 8B), suggesting that the LEV-mediated inhibition of SV2A protein does not operate in neuronal cells.

Next, in vitro cell-free protein stability assay was developed to test the hypothesis that LEV may cause the destabilization of SV2A protein in the lungs. Whole protein extracts prepared from the lungs of WT mice were incubated in the absence or presence of LEV at various concentrations at 37° C. for 1 hr, followed by immunoblot analysis of the reaction mixtures with anti-SV2A antibody. The levels of SV2A protein were significantly reduced in the presence of LEV as compared to that of the LEV-free negative control, suggesting that the binding of LEV to SV2A reduced the stability of SV2A protein under the in vitro cell-free incubation conditions (FIG. 8C).

In contrast, LEV failed to reduce SV2A protein present in brain extracts under similar conditions (FIG. 8D). These results recapitulated the differential effect of in vivo LEV treatment on SV2A protein expression in the lungs and the brain. There are two likely possibilities that may explain the differential effect of LEV on SV2A protein stability. The destruction pathway involved in the LEV-mediated degradation of SV2A protein may function specifically in the lungs but not in the brain. Alternatively, the intrinsic biochemical properties of SV2A proteins present in the lungs vs in the brain may be different (e.g. distinct posttranslational modifications), resulting in the differential effect of LEV on the stability of SV2A. These two models were tested using the in vitro cell-free protein stability assays. The inventors took advantage of the fact that SV2A protein in brain extract is at least 100-fold more enriched than in the lungs (FIG. 1A). We prepared two groups of reaction mixtures by mixing 0.2 μg of brain protein extract from WT mice with an excessive amount of either brain protein extract or lung protein extract prepared from Sv2a^{-/-} pup.

Under such assay conditions, the stability of SV2A protein from WT mouse brain was predominantly affected by the type of protein extracts from Sv2a^{-/-} mice that were present in excess amounts in the reaction mixtures. As shown in FIG. 8D, the presence of various concentrations of LEV (0.1 mM, 0.5 mM, 10 mM) did not cause the degradation of brain SV2A protein when incubated with an excessive amount of brain protein extracts from Sv2a2^{-/-} pup. In contrast, brain SV2A protein was completely degraded in the presence of 10 mM LEV when incubated with an excessive amount of lung protein extracts from Sv2a^{-/-} pup. These results indicated that the tissue microenvironment, rather than the SV2A protein itself, contributes to the observed tissue specific effect of LEV on the stability of SV2A protein, and suggested that the destruction mechanism responsible for the LEV-triggered degradation of SV2A protein operates specifically in the lungs, but not in the brain.

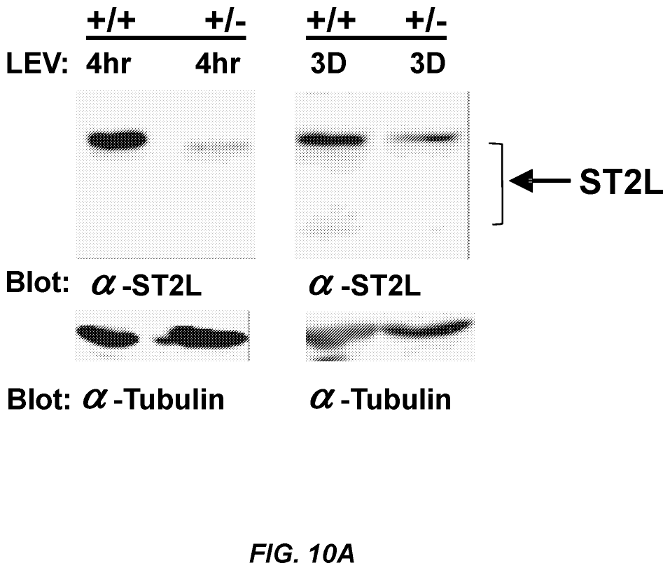

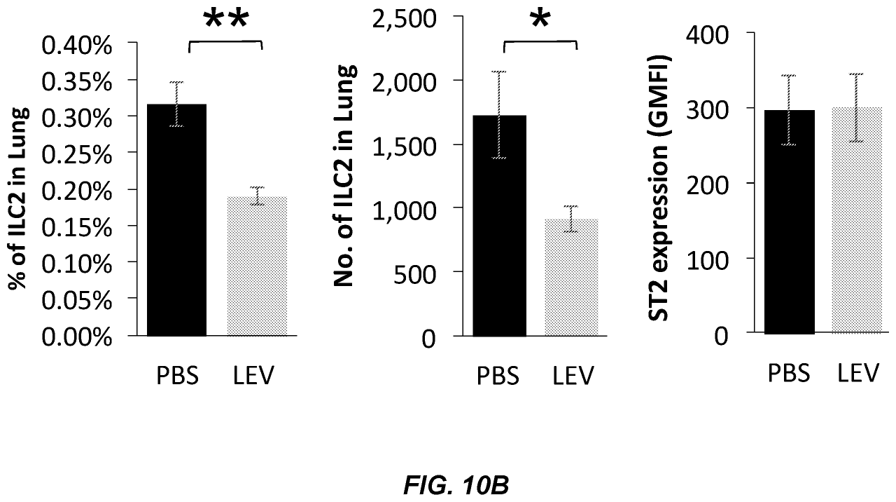

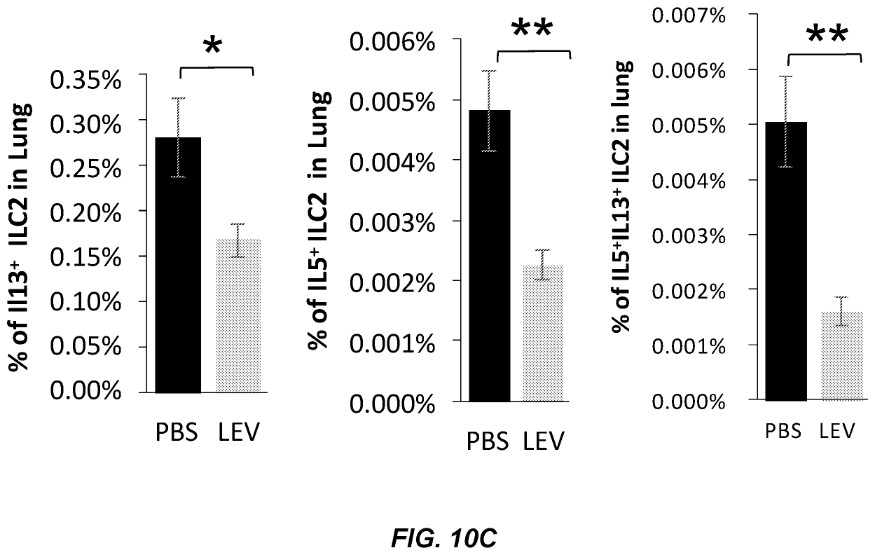

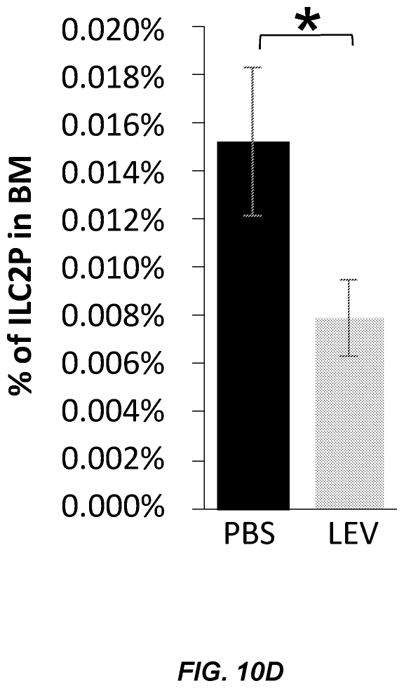

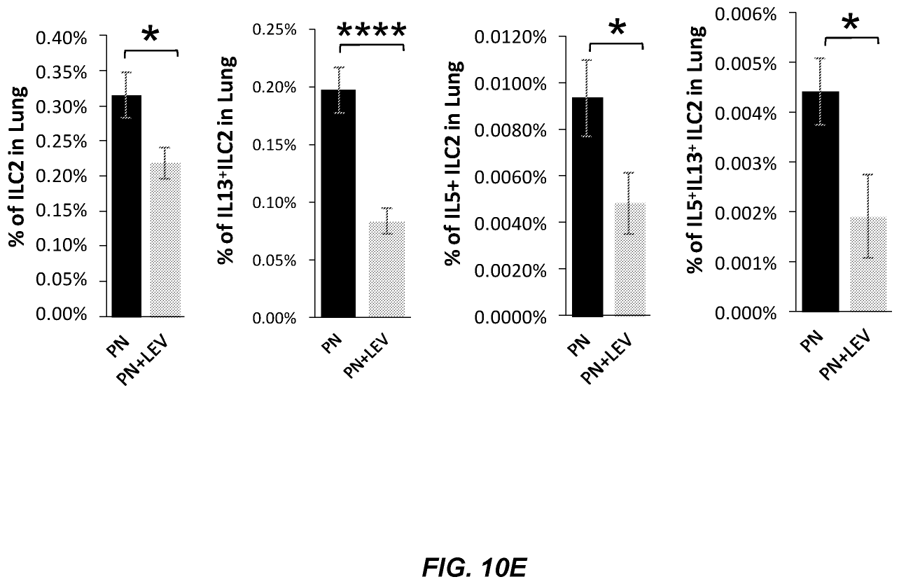

ILC2s play a key role in immune disorders and cancers, but no drugs have been approved for ILC2-based therapies. Since LEV causes the degradation of Sv2A, the inventors tested the hypothesis that LEV may mimic the effect of Sv2a disruption to suppress lung ST2L expression and ILC2 responses. Immunoblot analysis of protein extracts prepared from mice injected with PBS or LEV for 4 hr or daily for three consecutive days (3D) showed a clear suppression of ST2L protein (FIG. 10A). Flow cytometry analysis showed that LEV treatment caused about 50% reduction of the frequency and cell numbers of lung ILC2s (FIG. 10B) and suppressed the populations of lung ILC2 cells producing IL-13 and IL-5 (FIG. 10C). In addition, LEV treatment also suppressed the development of BM ILC2P (FIG. 10D), suggesting a stronger suppressive effect of LEV than Sv2a heterozygosity.

Next, the inventors tested whether LEV affects ILC2s under pathogenic conditions using a papain-induced mouse lung inflammation model in which ILC2 responses are triggered by papain administration. WT mice were challenged with papain in the presence or absence of LEV. As shown in FIG. 10E, LEV inhibited the papain-induced ILC2 responses by reducing the frequency of lung ILC2s and most significantly, the blocking of production of IL-5 and IL-13 by lung ILC2s. Taken together, these results indicated that LEV is an inhibitor of SV2A and has the ability to block lung ILC2 responses under pathogenic conditions.

In summary, these studies identified SV2A as a novel druggable target for the regulation of IL-33 signaling and ILC2 responses. As the proof of principle study, the FDA-approved anti-seizure drug LEV was shown to suppress IL-33 signaling and ILC2 responses in the lungs by targeting the SV2A-ST2 pathway.

### Example 2—Response to IL-33 in Sv2b^{+/-} mice

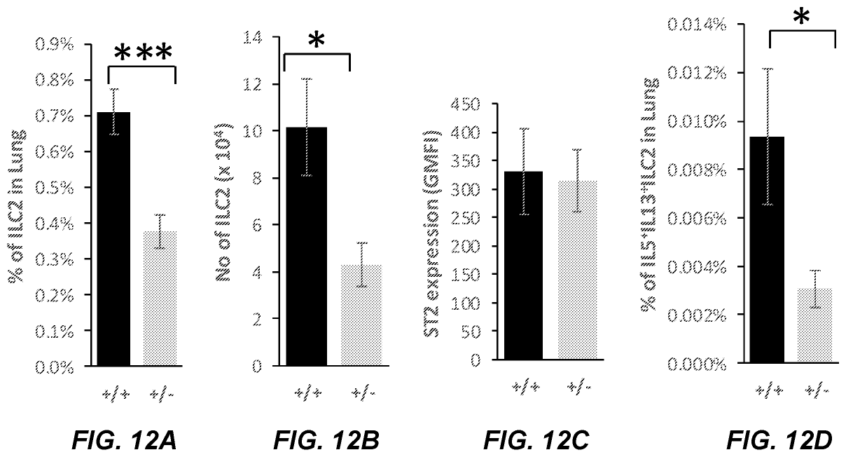

Sex-matched Sv2b^{+/+} and Sv2b^{+/-} littermates were intranasally injected with IL-33 (100 ng) daily on day 1, 2, 3, and lung tissue samples were harvested on day 4. The frequency (FIG. 12A) and numbers (FIG. 12B) of lung ILC2s (CD45⁺Lin⁻GATA3⁺CD90⁺CD25⁺), the geometric mean fluorescence intensity (GMFI) of ST2 in lung ILC2s (FIG. 12C), and the frequency of lung IL5⁺IL13⁺ILC2s (FIG. 12D) were analyzed by flow cytometry. Sv2b^{+/-} mice showed a reduction in frequency and numbers of lung ILC2s, as well as a reduction in frequency of lung IL5⁺IL13⁺ILC2s, following IL-33 treatment injection compared to Sv2b^{+/+} controls.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Burke, H. et al. Inflammatory phenotyping predicts clinical outcome in COVID-19. Respir Res 21, 245, doi:10.1186/s12931-020-01511-z (2020).
2. Xiong, Y. et al. Transcriptomic characteristics of bronchoalveolar lavage fluid and peripheral blood mononuclear cells in COVID-19 patients. Emerg Microbes Infect 9, 761-770, doi:10.1080/22221751.2020.1747363 (2020).
3. Wilkinson, T. et al. ACCORD: A Multicentre, Seamless, Phase 2 Adaptive Randomisation Platform Study to Assess the Efficacy and Safety of Multiple Candidate Agents for the Treatment of COVID-19 in Hospitalised Patients: A structured summary of a study protocol for a randomised controlled trial. Trials 21, 691, doi:10.1186/s13063-020-04584-9 (2020).
4. Zizzo, G. & Cohen, P. L. Imperfect storm: is interleukin-33 the Achilles heel of COVID-19? Lancet Rheumatol, doi:10.1016/S2665-9913 (20) 30340-4 (2020).
5. Stanczak, M. A. et al. IL-33 expression in response to SARS-COV-2 correlates with seropositivity in COVID-19 convalescent individuals. medRxiv, published online July 10, doi: https://doi.org/10.1101/2020.07.09.20148056_ (2020).
6. Kakkar, R. & Lee, R. T. The IL-33/ST2 pathway: therapeutic target and novel biomarker.Nat Rev Drug Discov 7, 827-840, doi:10.1038/nrd2660 (2008).
7. Martin, N. T. & Martin, M. U. Interleukin 33 is a guardian of barriers and a local alarmin.Nat Immunol 17, 122-131, doi:10.1038/ni.3370 (2016).
8. Pascual-Figal, D. A. & Januzzi, J. L. The biology of ST2: the International ST2 Consensus Panel. Am J Cardiol 115, 3B-7B, doi:10.1016/j.amjcard.2015.01.034 (2015).
9. Liew, F. Y., Girard, J. P. & Turnquist, H. R. Interleukin-33 in health and disease. Nat Rev Immunol 16, 676-689, doi:10.1038/nri.2016.95 (2016).
10. Barlow, J. L. & Mckenzie, A. N. J. Innate Lymphoid Cells of the Lung. Annu Rev Physiol 81, 429-452, doi:10.1146/annurev-physiol-020518-114630 (2019).
11. Vasanthakumar, A. & Kallies, A. Interleukin (IL)-33 and the IL-1 Family of Cytokines-Regulators of Inflammation and Tissue Homeostasis. Cold Spring Harb Perspect Biol 11, doi:10.1101/cshperspect.a028506 (2019).
12. Hatzioannou, A. et al. An intrinsic role of IL-33 in Treg cell-mediated tumor immunoevasion. Nat Immunol 21, 75-85, doi:10.1038/s41590-019-0555-2 (2020).
13. Schuijs, M. J. et al. ILC2-driven innate immune checkpoint mechanism antagonizes NK cell antimetastatic function in the lung. Nat Immunol 21, 998-1009, doi:10.1038/s41590-020-0745-y (2020).
14. Taniguchi, S. et al. Tumor-initiating cells establish an IL-33-TGF-beta niche signaling loop to promote cancer progression. Science 369, doi: 10.1126/science.aayl813 (2020).
15. Moral, J. A. et al. ILC2s amplify PD-1 blockade by activating tissue-specific cancer immunity. Nature 579, 130-135, doi:10.1038/s41586-020-2015-4 (2020).
16. Bartholome, O. et al. Puzzling Out Synaptic Vesicle 2 Family Members Functions. Front Mol Neurosci 10, 148, doi:10.3389/fnmol.2017.00148 (2017).
17. Stout, K. A., Dunn, A. R., Hoffman, C. & Miller, G. W. The Synaptic Vesicle Glycoprotein 2: Structure, Function, and Disease Relevance. ACS Chem Neurosci 10, 3927-3938, doi:10.1021/acschemneuro.9b00351 (2019).
18. Loscher, W., Gillard, M., Sands, Z. A., Kaminski, R. M. & Klitgaard, H. Synaptic Vesicle Glycoprotein 2A Ligands in the Treatment of Epilepsy and Beyond. CNS Drugs 30, 1055-1077, doi:10.1007/s40263-016-0384-x (2016).
19. Vainchtein, I. D. et al. Astrocyte-derived interleukin-33 promotes microglial synapse engulfment and neural circuit development. Science 359, 1269-1273, doi:10.1126/science.aal3589 (2018).
20. Fairlie-Clarke, K. et al. Expression and Function of IL-33/ST2 Axis in the Central Nervous System Under Normal and Diseased Conditions. Front Immunol 9, 2596, doi:10.3389/fimmu.2018.02596 (2018).
21. Janz, R., Goda, Y., Geppert, M., Missler, M. & Sudhof, T. C. SV2A and SV2B function as redundant Ca2+ regulators in neurotransmitter release. Neuron 24, 1003-1016 (1999).
22. Crowder, K. M. et al. Abnormal neurotransmission in mice lacking synaptic vesicle protein 2A (SV2A). Proc Natl Acad Sci U S A 96, 15268-15273 (1999).
23. Espinassous, Q. et al. IL-33 enhances lipopolysaccharide-induced inflammatory cytokine production from mouse macrophages by regulating lipopolysaccharide receptor complex. J Immunol 183, 1446-1455, doi: 10.4049/jimmunol.0803067 (2009).
24. Tago, K. et al. ST2 gene products critically contribute to cellular transformation caused by an oncogenic Ras mutant. Heliyon 3, e00436, doi:10.1016/j.heliyon.2017.e00436 (2017).
25. Tominaga, S. A putative protein of a growth specific cDNA from BALB/c-3T3 cells is highly similar to the extracellular portion of mouse interleukin 1 receptor. FEBS Lett 258, 301-304, doi: 10.1016/0014-5793 (89) 81679-5 (1989).
26. Klemenz, R., Hoffmann, S. & Werenskiold, A. K. Serum-and oncoprotein-mediated induction of a gene with sequence similarity to the gene encoding carcinoembryonic antigen. Proc Natl Acad Sci U S A 86, 5708-5712, doi:10.1073/pnas.86.15.5708 (1989).
27. Yanagisawa, K., Takagi, T., Tsukamoto, T., Tetsuka, T. & Tominaga, S. Presence of a novel primary response gene ST2L, encoding a product highly similar to the interleukin 1 receptor type 1. FEBS Lett 318, 83-87, doi:10.1016/0014-5793 (93) 81333-u (1993).
28. de Kleer, I. M. et al. Perinatal Activation of the Interleukin-33 Pathway Promotes Type 2 Immunity in the Developing Lung. Immunity 45, 1285-1298, doi:10.1016/j.immuni.2016.10.031 (2016).
29. Saluzzo, S. et al. First-Breath-Induced Type 2 Pathways Shape the Lung Immune Environment. Cell Rep 18, 1893-1905, doi:10.1016/j.celrep.2017.01.071 (2017).
30. Loering, S., Cameron, G. J. M., Starkey, M. R. & Hansbro, P. M. Lung development and emerging roles for type 2 immunity. J Pathol 247, 686-696, doi:10.1002/path.5211 (2019).
31. Barlow, J. L. et al. IL-33 is more potent than IL-25 in provoking IL-13-producing nuocytes (type 2 innate lymphoid cells) and airway contraction. J Allergy Clin Immunol 132, 933-941, doi:10.1016/j.jaci.2013.05.012 (2013).
32. Neill, D. R. et al. Nuocytes represent a new innate effector leukocyte that mediates type-2 immunity. Nature 464, 1367-1370, doi:10.1038/nature08900 (2010).
33. Klitgaard, H. & Verdru, P. Levetiracetam: the first SV2A ligand for the treatment of epilepsy. Expert Opin Drug Discov 2, 1537-1545, doi:10.1517/17460441.2.11.1537 (2007).
34. Gillard, M., Chatelain, P. & Fuks, B. Binding characteristics of levetiracetam to synaptic vesicle protein 2A (SV2A) in human brain and in CHO cells expressing the human recombinant protein. Eur J Pharmacol 536, 102-108, doi:10.1016/j.ejphar.2006.02.022 (2006).
35. Tahk, S. et al. Control of specificity and magnitude of NF-KB and STAT1-mediated gene activation through PIASy and PIAS1 cooperation. Proc. Natl Acad. Sci. USA 104, 11643-11648 (2007).
36. Duerr, C. U. & Fritz, J. H. Isolation of Group 2 Innate Lymphoid Cells from Mouse Lungs. Methods Mol Biol 1656, 253-261, doi:10.1007/978-1-4939-7237-1_16 (2017).
37. Hoyler, T. et al. The transcription factor GATA-3 controls cell fate and maintenance of type 2 innate lymphoid cells. Immunity 37, 634-648, doi: 10.1016/j.immuni.2012.06.020 (2012).
38. Liu, B. et al. PIAS1 selectively inhibits interferon-inducible genes and is important in innate immunity. Nat. Immunol. 5, 891-898 (2004).
39. Liu, B. et al. Proinflammatory stimuli induce IKKalpha-mediated phosphorylation of PIAS1 to restrict inflammation and immunity. Cell 129, 903-914 (2007).

What is claimed:

1. A method for treating a subject for a condition associated with group 2 innate lymphoid cells (ILC2s), the method comprising administering to the subject an effective amount of an agent that reduces an amount of a synaptic vesicle glycoprotein 2 (SV2) protein.

2. The method of claim 1, wherein the SV2 protein is SV2A.

3. The method of claim 1, wherein the SV2 protein is SV2B.

4. The method of claim 1, wherein the SV2 protein is SV2C.

5. The method of any of claims 1-4, wherein the condition is an autoimmune condition.

6. The method of any of claims 1-4, wherein the condition is an allergic disorder.

7. The method of any of claims 1-4, wherein the condition is cancer.

8. The method of any of claims 1-4, wherein the condition is pneumonia.

9. The method of claim 8, wherein the condition is COVID-19 pneumonia.

10. The method of any of claims 1-9, wherein the agent increases a rate of degradation of the SV2 protein.

11. The method of any of claims 1-9, wherein the agent decreases an expression level of an SV2 mRNA encoding the SV2 protein.

12. The method of any of claims 1-11, wherein the agent is a nucleic acid targeting agent.

13. The method of claim 12, wherein the nucleic acid targeting agent is an siRNA, an shRNA, or an antisense oligonucleotide.

14. The method of any of claims 1-11, wherein the agent is an SV2A-binding agent.

15. The method of claim 14, wherein the SV2A-binding agent is levetiracetam.

16. The method of claim 15, wherein levetiracetam is administered to the subject at a dose of between 250 mg and 1000 mg per day for two or more consecutive days.

17. The method of claim 14, wherein the SV2A-binding agent is brivaracetam.

18. The method of claim 17, wherein brivaracetam is administered to the subject at a dose of between 50 mg and 200 mg for two or more consecutive days.

19. The method of any of claims 1-18, wherein administering the effective amount of the agent reduces an amount of a cell surface protein in cells of the subject.

20. The method of claim 19, wherein the cell surface protein is a cell surface receptor.

21. The method of claim 19 or 20, wherein the cell surface protein is ST2L.

22. The method of any of claims 1-21, wherein administering the effective amount of the agent reduces a number of ILC2s in the subject.

23. The method of claim 19 or 20, wherein the cell surface protein is ACE2.

24. The method of claim 19 or 20, wherein the cell surface protein is PD-1.

25. A method for reducing an amount of a cell surface receptor in a cell comprising administering to the cell an agent that reduces an amount of an SV2 protein in the cell.

26. The method of claim 25, wherein the SV2 protein is SV2A.

27. The method of claim 25, wherein the SV2 protein is SV2B.

28. The method of claim 25, wherein the SV2 protein is SV2C.

29. The method of any of claims 25-28, wherein the agent increases a rate of degradation of the SV2 protein.

30. The method of any of claims 25-28, wherein the agent decreases an expression level of an SV2 mRNA encoding the SV2 protein.

31. The method of any of claims 25-30, wherein the agent is a nucleic acid targeting agent.

32. The method of claim 31, wherein the agent is an siRNA, an shRNA, or an antisense oligonucleotide.

33. The method of any of claims 25-30, wherein the agent is an SV2A-binding agent.

34. The method of claim 33, wherein the agent is levetiracetam or a derivative thereof.

35. The method of claim 33, wherein the agent is brivaracetam or a derivative thereof.

36. The method of any of claims 25-35, wherein the cell surface receptor is ST2L.

37. The method of any of claims 25-35, wherein the cell surface protein is ACE2.

38. The method of any of claims 25-35, wherein the cell surface protein is PD-1.

39. A method for treating a subject for COVID-19 pneumonia, the method comprising administering to the subject an effective amount of levetiracetam or brivaracetam.

40. The method of claim 39, wherein administering to the subject the effective amount of levetiracetam or brivaracetam reduces an amount of an SV2 protein in cells of the subject.

41. The method of claim 40, wherein the SV2 protein is SV2A.

42. The method of claim 40, wherein the SV2 protein is SV2B.

43. The method of claim 40, wherein the SV2 protein is SV2C.

44. The method of any of claims 39-43, wherein administering to the subject the effective amount of levetiracetam or brivaracetam reduces a number of ILC2s in the subject.

45. The method of any of claims 39-44, wherein the method comprises administering to the subject an effective amount of levetiracetam.

46. The method of claim 45, wherein levetiracetam is administered to the subject at a dose of between 250 mg and 1000 mg per day for two or more consecutive days.

47. The method of any of claims 39-44, wherein the method comprises administering to the subject an effective amount of brivaracetam.

48. The method of claim 47, wherein brivaracetam is administered to the subject at a dose of between 50 mg and 200 mg for two or more consecutive days.

49. A method for treating a subject for cancer, the method comprising administering to the subject an effective amount of levetiracetam or brivaracetam.

50. The method of claim 49, wherein administering to the subject the effective amount of levetiracetam or brivaracetam reduces an amount of an SV2 protein in cells of the subject.

51. The method of claim 50, wherein the SV2 protein is SV2A.

52. The method of claim 50, wherein the SV2 protein is SV2B.

53. The method of claim 50, wherein the SV2 protein is SV2C.

54. The method of any of claims 49-53, wherein administering to the subject the effective amount of levetiracetam or brivaracetam reduces a number of ILC2s in the subject.

55. The method of any of claims 49-54, wherein the method comprises administering to the subject an effective amount of levetiracetam.

56. The method of claim 55, wherein levetiracetam is administered to the subject at a dose of between 250 mg and 1000 mg per day for two or more consecutive days.

57. The method of any of claims 49-54, wherein the method comprises administering to the subject an effective amount of brivaracetam.

58. The method of claim 57, wherein brivaracetam is administered to the subject at a dose of between 50 mg and 200 mg for two or more consecutive days.

59. A method for treating a subject for an autoimmune disorder, the method comprising administering to the subject an effective amount of levetiracetam or brivaracetam.

60. The method of claim 59, wherein administering to the subject the effective amount of levetiracetam or brivaracetam reduces an amount of an SV2 protein in cells of the subject.

61. The method of claim 60, wherein the SV2 protein is SV2A.

62. The method of claim 60, wherein the SV2 protein is SV2B.

63. The method of claim 60, wherein the SV2 protein is SV2C.

64. The method of any of claims 59-63, wherein administering to the subject the effective amount of levetiracetam or brivaracetam reduces a number of ILC2s in the subject.

65. The method of any of claims 59-64, wherein the method comprises administering to the subject an effective amount of levetiracetam.

66. The method of claim 65, wherein levetiracetam is administered to the subject at a dose of between 250 mg and 1000 mg per day for two or more consecutive days.

67. The method of any of claims 59-64, wherein the method comprises administering to the subject an effective amount of brivaracetam.

68. The method of claim 67, wherein brivaracetam is administered to the subject at a dose of between 50 mg and 200 mg for two or more consecutive days.

69. A method for increasing an amount of a cell surface receptor in a cell comprising administering to the cell an agent that increases an amount of an SV2 protein in the cell.

70. The method of claim 69, wherein the SV2 protein is SV2A.

71. The method of claim 69, wherein the SV2 protein is SV2B.

72. The method of claim 69, wherein the SV2 protein is SV2C.

73. The method of any of claims 69-72, wherein the agent decreases a rate of degradation of the SV2 protein.

74. The method of any of claims 69-72, wherein the agent increases an expression level of an SV2 mRNA encoding the SV2 protein.

75. The method of any of claims 69-74, wherein the agent is an SV2A-binding agent.

76. The method of any of claims 69-75, wherein the cell surface receptor is ST2L.

77. The method of any of claims 69-75, wherein the cell surface protein is ACE2.

78. The method of any of claims 69-75, wherein the cell surface protein is PD-1.
